(19) United States
(12) Patent Application Publication
(10) Pub. No.: US 2013/0058929 A1
(43) Pub. Date: Mar. 7, 2013

(54) FUSION PROTEINS FOR INHIBITION AND DISSOLUTION OF COAGULATION OF THROMBI

(71) Applicants: VLADIMIR R. MUZYKANTOV, Warwick, PA (US); Claudia Gottstein, Carpinteria, CA (US); Bi-Sen Ding, New York, NY (US); Douglas B. Cines, Wynnewood, PA (US)

(72) Inventors: VLADIMIR R. MUZYKANTOV, Warwick, PA (US); Claudia Gottstein, Carpinteria, CA (US); Bi-Sen Ding, New York, NY (US); Douglas B. Cines, Wynnewood, PA (US)

(73) Assignee: The Trustees of the University of Pennsylvania, Philadelphia, PA (US)

(21) Appl. No.: 13/528,125

(22) Filed: Jun. 20, 2012

Related U.S. Application Data
(63) Continuation of application No. 12/089,250, filed on Oct. 21, 2008, now abandoned.
(60) Provisional application No. 60/723,899, filed on Oct. 5, 2005.

(51) Int. Cl. A61K 39/395; A61P 7/02

(52) U.S. Cl. 424/134.1

(57) ABSTRACT

Fusion proteins containing a ligand which specifically binds to a selected vascular bed linked to an anti-thrombotic molecule are provided. Also provided are methods for use of these fusion proteins to prevent coagulation, to dissolve blood clots and to protect against the risk of iatrogenic side effects including those arising from cancer therapy and specific vascular occluding agents.

### CROSS-REFERENCE TO RELATED APPLICATIONS

This is a continuation of U.S. patent application Ser. No. 12/089,250, filed Apr. 4, 2008, which is a national stage application under 35 U.S.C. 371 of PCT/US2006/038989, filed Oct. 5, 2006, which claims the benefit under 35 USC 119(e) of U.S. Patent Application No. 60/723,899, filed Oct. 5, 2005, which applications are incorporated by reference herein.

### STATEMENT REGARDING FEDERALLY SPONSORED RESEARCH OR DEVELOPMENT

This invention was made with government support under grant No. DAMD17-02-1-0197 awarded by the Department of Defense. The government has certain rights in the invention.

### FIELD OF THE INVENTION

The present invention relates to compositions comprising fusion proteins, preferably recombinant fusion proteins, which represent a continuous polypeptide chain combining distinct anti-thrombotic and targeting entities, which do not exist naturally as a single entity but rather as separate and distinct entities, and methods for use of these compositions to inhibit thrombosis and/or dissolve clots in the vasculature. Compositions of the present invention are thus useful in prevention and treatment of all pathologic conditions associated with an increased risk of thrombosis including, but in no way limited to, pulmonary embolism, myocardial infarction, stroke and iatrogenic or spontaneous thrombosis. Compositions of the present invention are also useful as protective agents in therapies wherein specific vascular occluding agents are administered, as well as in other medical interventions associated with a high risk of iatrogenic thrombosis.

### BACKGROUND OF THE INVENTION

Prophylactic and therapeutic use of existing anti-thrombotic agents including anti-coagulants, anti-platelet agents, and fibrinolytic plasminogen activators is greatly limited by inadequate delivery in the vasculature and lack of durable, specific and safe effects of these agents in the blood stream.

In particular, numerous previous attempts have been described to design new improved versions of plasminogen activators. Recombinant plasminogen activators with deleted and mutated domains, possessing, as a result of these molecular modifications, greater enzymatic potency, resistance to plasma inhibitors, and slightly more prolonged longevity in circulation (e.g., half-life extension from 5 minutes to 25 minutes) have been described. For example, in the quest for more specific and safe plasminogen activator therapies, a thrombin-activated uPA variant (lmw-scuPA-T) in which the endogenous plasmin-sensitive activation site was replaced by one cleaved by thrombin was described (Yang et al. Biochemistry 1994 33:p 606-612). However, clinical studies showed that none of these derivatives produced decisively better therapies.

Further attempts have been made to target anti-thrombotic agents to the blood clots via fusion proteins comprising anti-thrombotic agents. In particular, fusion proteins have been described containing a fibrin or fibrinogen specific antibody or a fragment thereof and urokinase or hirudin (Holvoet et al. Eur. J. Biochem. 210:945-52; Wang, X. and Yu, W. Chin. J. Biotechnol. 1999 15:23-28; Liu et al. Sheng Wu Gong Cheng Xue Bang 2002 18:509-11; Peter et al. Circulation 2000 101:1158-64) or tissue plasminogen activator (tPA) or urokinase (Love et al. Thrombosis Res. 2:211-9; Runge et al. Mol. Biol. & Med. 8(2):245-255). These recombinant proteins bind wherever fibrin is present.

However, these targeting moieties are not designed for thromboprophylaxis or prevention of clot formation and/or coagulation since their target does not exist prior to thrombosis. Further, inability of fusion proteins comprising fibrinolytic agents to permeate into the fibrin meshwork has hindered their efficacy at therapeutic dissolution of blood clots formed prior to the intervention. However, this type of administration, i.e. emergency injection of fibrinolytic plasminogen activators as soon as possible post-thrombosis, is the only currently employed used of fibrinolytics.

In general, this type of fibrinolysis, i.e., post-event therapy, is marred by inevitable delays (time needed for diagnosis, transportation, injection and the lysis proper, slowed by poor clot permeability), causing ischemia-reperfusion injury that worsens outcome.

Further, it is only in very rare cases that thrombosis represents an isolated single event. In essence, initial thrombotic event in most cases is a manifestation of a general imbalance of coagulation and hemostasis predisposing the patient to recurrences. Further, vascular trauma, ischemia and inflammation caused by initial thrombosis greatly further predispose to and even provoke secondary, tertiary and subsequent thrombotic events. Finally, most of patient are immobilized, weakened or otherwise adversely affected in the post-thrombotic period, which leads to blood stasis (e.g., in the extremities, DVT) and re-thromboses.

Thrombosis recurrence, therefore, represents a major health problem and adequate thromboprophylaxis represents an unmet medical need. For example, approximately 20% patients with an initially favorable response to tPA develop symptomatic re-occlusion and 4-15% of patients with transient ischemic attacks and myocardial infarction develop a stroke within hours to days of presentation. Current recommendations for management of patients with transient ischemic attack, unstable angina and other acute cardiovascular disorders include immediate hospitalization for several days, in particular to enable doctors to apply emergency anti-thrombotic treatment in these predisposed patients as soon as possible if symptoms of thrombosis occur.

Situations in which a patient is at highest risk for occurrence or recurrence of thrombosis and means to identify such high-risk patients are known. However, current anticoagulant and anti-platelet agents provide only modest prophylaxis against recurrence or new strokes because they are non-targeted and therefore have to be administered at doses which are below the optimal biological effective doses in order to avoid serious side effects such as described further herein. Therefore, the majority of at-risk patients are not adequately protected.

Recombinant proteins have also been targeted to blood cells, e.g. activated platelets via the target P-selectin (Wan et al. Thromb Res. 2000 97(3):133-41) or monocytes and neutrophils via P-selectin ligand (Fujise et al. Circulation 1997 95:715-22). Utility of these targeted constructs is limited as in the anti-fibrin case described above, since the target does not exist before thrombosis. Further, once these blood cells are lodged into a clot, they have limited accessibility. In addition, targeting to blood cells lacks specificity to selected vascular sites because the cells are circulating systemically in the blood.

Systemic circulation of anti-thrombotic agents has a danger of side effects, including permeation into extravascular space, where the anti-thrombotic agent can inflict collateral damage including, but not limited to, pathological remodeling of extracellular matrix, neurotoxicity and activation of pro-inflammatory cells, proteases and growth factors. In theory, anchoring of anti-thrombotic agents on the surface of endothelial cells lining vascular lumen may help to restrict these side effects and augment thromboprophylaxis, due to prevention of formation or/and swift dissolution of nascent blood clots formed within or lodging as emboli into such pre-conditioned vessels expressing anti-thrombotic agents in the lumen. Maintaining activity of the anti-thrombotic agent for prolonged periods in the bloodstream is a challenge for these types of constructs as well, since it is well known that anticoagulants and fibrinolytics undergo inactivation and elimination in the bloodstream.

Targeting of anti-thrombotic agents to endothelial surfaces in selected vascular sites (e.g. deep veins of lower extremities, cranial arteries, pulmonary arteries and capillaries) is also desirable since systemic side effects, e.g. bleeding or organ specific toxicities induced by freely circulating drugs limit their utility. It is essential, that the effector moiety remains active after fusing it to the targeting moiety; thus the targeting moiety must not interfere with activity of the anti-thrombotic agent.

Therapeutic approaches for neoangiogenic diseases, e.g. cancer and proliferative retinopathy, have been suggested which selectively occlude tumor or ocular blood vessels by specific activation of the coagulation system (Huang et al. Science 1997 275:547-550; Birchler et al. Nature Biotechnol 1999; 17:984-988; Dienst et al. J. Natl Cancer Inst. 2005 97:7333-747; Narazaki and Tosato J. Natl Cancer Inst. 2005 97:705-707). This approach has been termed specific vascular occlusion (SVO). It is also desirable in these therapies to protect non-target vasculature from coagulation induction during SVO treatment cycles. Thus, in this therapeutic approach, antithrombotic therapy should be targeted to selected vascular sites of risk, which differ from those treated with SVO agents, in order to not counteract the coagulation induction in the vessels treated with SVO agents.

The search for new luminal endothelial cell surface markers has led to the identification of vascular addresses, and new technologies which may allow targeting of organ specific vascular beds (Ruoslahti, E Biochemical Society Transactions 2004 32: 397-402; Arap et al. Nature Medicine 2002 8: 121-7; and Oh et al. Nature 2004; 429: 629-35.

Biochemical conjugation of a drug to anti-PECAM, also known as anti-CD31 to facilitate delivery of a drug into the endothelium is disclosed in PCT Application PCT/US99/05279. A PECAM antibody biochemically conjugated with streptavidin has also been disclosed as a carrier for delivery of drugs into the endothelium (Muzykantov et al. (Am. J. Resp. Crit. Care Med. 1998 157:A₂O₃). Biochemical conjugates of tissue plasminogen activator with antibodies to angiotensin-converting enzyme (Murciano et al. Am. J. Resp. Crit. Care Med. 2001 164:1295-1302) and inter-cellular adhesion molecule (Murciano et al. Blood 2003 101:3977-84) and urokinase with antibodies to RE85F (an antigen on rat lung endothelium; Ding et al. Circulation 2003 108:r129-r135) and glycoprotein IIb/IIIa (More et al. Cardiovasc. Res. 1993 27:2200-4) have also been disclosed.

While these agents are effective at targeting the drug to the vascular bed in animal studies, biochemical conjugates are of limited interest pharmaceutically because generally they cannot be produced as homogeneous substances, thus impeding manufacturing, quality control and administration. Further, it is extremely difficult to obtain monovalent conjugates by biochemical conjugation, even for the laboratory use. However, conjugates containing polyvalent anti-PECAM or anti-ICAM undergo internalization and disappear from the lumen, thus rendering such conjugates ineffective in terms of anti-thrombotic interventions.

In the present invention fusion proteins combining anti-thrombotic and targeting agents, which otherwise exist naturally only as separate and distinct entities, are provided which target an anti-thrombotic molecule to the endothelial luminal surface in the vascular bed while maintaining its anti-thrombotic activity.

### SUMMARY OF THE INVENTION

An object of the present invention is to provide a series of fusion protein constructs comprising a ligand, which specifically binds to a surface determinant on vascular endothelium, linked to a fibrinolytic or coagulation-inhibiting effector or anti-thrombotic molecule in a form of a continuous polypeptide chain combining anti-thrombotic and targeting agents, which otherwise exist naturally only as separate and distinct entities.

Another object of the present invention is to provide a fusion protein as described above, which binds to an endothelial cell surface determinant and remains exposed on the vascular lumen thereby acting in the blood.

Another object of the present invention is to provide a fusion protein as described above, which is activated locally at a site of a pathological process such as a site of active thrombosis by a pathological factor such as thrombin presented at the site.

Another object of the present invention is to provide a fusion protein as described above, which does not interact with its plasma inhibitors prior to activation by thrombin or other factors presented in the site of thrombosis.

Another object of the present invention is to provide a method for inhibiting coagulation in a vascular bed of interest that comprises administering a fusion protein comprising a ligand, which specifically binds to endothelial cells in the vascular bed, linked to an effector inhibiting activation of the coagulation cascade or of platelets.

Another object of the present invention is to provide a method for local release of an anti-thrombotic agent from endothelium-anchored fusion protein by proteolytic cleavage of a specific site in the ligand and/or fibrinolytic effector and/or their linker sensitive to a protease that is active only in the sites of active thrombosis.

Another object of the present invention is to provide a method of dissolving blood clots in a vascular bed of interest that comprises administering a fusion protein comprising a ligand, which specifically binds to endothelial cells in the vascular bed, linked to a fibrinolytic effector.

Another object of the present invention is to provide a method for dissolving blood clots in a selected vascular bed of an animal comprising prophylactically administering to an animal diagnosed as being predisposed or at high risk for thrombosis or thrombosis recurrence a fusion protein comprising a ligand, which specifically binds to endothelial cells in the vascular bed, linked to a fibrinolytic effector.

Yet another object of the present invention is to provide a method for attenuation of side effects of medical interventions associated with a potentially increased risk of iatrogenic thrombosis including that following administration of a specific vascular occluding agent in cancer therapy. This method comprises coadministering with the specific vascular occluding agent a fusion protein comprising a ligand, which binds to an endothelial surface determinant, linked to a fibrinolytic or coagulation-inhibiting effector.

### BRIEF DESCRIPTION OF THE FIGURES

FIG. 1 provides a diagram depicting an embodiment of a recombinant fusion protein of the present invention specifically binding to an endothelium specific antigen. As shown in the diagram, precursor of an anti-thrombotic protein depicted as substance 1 is activated by the effector of the recombinant fusion protein to substance 2, which inhibits coagulation or initiates fibrinolysis.

FIG. 2 is a diagram illustrating the generation of a scFv molecule. This is the first step and precondition for creating various fusion proteins according to this invention. Agarose gel electrophoresis shows the RNA and DNA encoding for variable light and heavy chains (VH and VL) of the antibody 390 (anti-murine-PECAM-antibody), which were prepared from the corresponding hybridoma cell line. The VH and VL fragments were then fused to a scFv, as evidenced by the third electrophoresis gel and cloned into various plasmids.

FIG. 3 provides histograms from a flow cytometry analysis, which compares the binding activity of the scFv to its parental IgG antibody. The scFv (lower panel) tagged with soluble tissue factor (sTF) binds specifically to PECAM expressing mouse endothelial cells with the same affinity as the parental IgG (upper panel). sTF tag alone did not bind to the endothelial cells. Thin lines represent cells only; dotted lines represent cells with secondary and tertiary antibody; bold lines represent cells incubated with tagged scFv and secondary/tertiary antibody. The experiment confirms the correct cloning and functional expression of the scFv.

FIG. 4 shows an agarose electrophoresis gel detecting DNA bands of the correct size (1059 bp) for the kringle 2 and protease domain of human tPA, illustrating the successful cloning of a suitable effector moiety from human endothelial cells. The DNA was amplified by PCR (polymerase chain reaction) from human endothelial cells after preparing RNA and cDNA as described in FIG. 2. Lanes show PCR amplificates from cDNA of 1: human leukemia cells; 2 and 3: human arterial endothelial cells; 4: human venous endothelial cells. Lane 5 is a reamplification PCR reaction from DNA that was originally amplified from human arterial endothelial cells. Lane 6 is a DNA size standard (hundred base pair ladder).

FIG. 5A shows the design of the expression vector pCG-F1 for expression of fusion proteins in prokaryotic cells, consisting of a scFv against an endothelial cell marker, and the kringle 2 and protease domain of tPA. The restriction sites directly upstream of VH (variable heavy chain region) and directly downstream of VL (variable light chain region) of the scFv allow easy exchange of scFv cassettes. In this example, a scFv directed against murine PECAM1 was incorporated into pCG-F1, and the biochemical characterization of the resulting fusion protein is shown in panels B through E. Arrows in B through E point at the 70 kDA position in the gels or blot.

FIG. 5B is a western blot of fusion protein detected with an antibody against human tPA, confirming the identity of the protein seen in FIGS. 5C and 5D.

FIG. 5C is a SDS-PAGE of fusion protein stained with Coomassie Blue, demonstrating a single band at 70 kDA, and a very faint band at approximately 140 kDA, potentially a dimer form of tPA. The amount is negligible.

FIG. 5D is a SDS-PAGE of fusion protein stained with a very sensitive Silverstain, confirming that there are no other bands present than the ones seen in FIG. 5C.

FIG. 5E shows a spectrophotometric scan of the fusion protein, demonstrating a typical protein profile (peak at 278 nm, shoulder after 280 nm) without contaminants.

FIG. 6 is a line graph showing dose-dependent activation of plasminogen to plasmin by the fusion protein anti-CD31-tPA. Two experiments are shown. The upper two curves show plasminogen conversion in the presence of soluble fibrin while the lower two curves show plasminogen conversion in the absence of soluble fibrin. This confirms that the increase in activity conferred by fibrin has been retained in the recombinant tPA.

FIG. 7A through 7D show the molecular design, cloning and biochemical characterization of a second exemplary fusion protein of this invention. This fusion protein comprises an anti-PECAM scFv such as described in FIGS. 2 and 3 and low molecular weight single chain urokinase-like plasmin activator (lmw scuPA).

FIG. 7A is a schematic diagram describing the expression vector pMT-BD1 for expression of the fusion protein in mammalian cells. In this embodiment, the anti PECAM scFv is fused to the N-terminus of lmw-scuPA by a linker SSSSGSSSSGAAA (SEQ ID NO:6). As shown in this figure, the variable regions of heavy and light chain (VH and VL) were fused to the synthetic scFv-antibody fragment using a (Gly₄Ser)₃ linker sequence (GGGGSGGGGSGGGGS; SEQ ID NO:11).

FIG. 7B is an agarose electrophoresis gel evidencing ligation and cloning of lmw-scuPA and anti-PECAM scFv derived from cloning vectors into SpeI and XhoI sites of the pMT expression vector via XhoI and SpeI digestion of the fusion construct.

FIG. 7C shows a western blot analysis of 40 μl of culture medium alone or after induction by 0.5 mM CuSO₄. Purified fusion protein (50 ng and 200 ng) was blotted to compare expression levels.

FIG. 7D shows a 10-15% gradient SDS-PAGE of purified fusion protein with or without plasmin treatment under unreduced or reduced conditions.

FIG. 8A through 8C provide evidence of specific binding of scFv-uPA fusion protein to cells expressing mouse PECAM.

FIG. 8A shows FITC-streptavidin staining of REN/PECAM (left) versus control REN (right) cells after incubation with biotinylated anti-PECAM scFv-scuPA (40× magnification).

FIG. 8B is a line graph showing results from an ELISA measuring binding of anti-PECAM scFv-scuPA fusion protein to REN/PECAM (closed circles) versus REN (open circles) cells.

FIG. 8C is a line graph showing results from an ELISA measuring inhibition of binding of fusion protein to REN/PECAM cells by parental anti-PECAM IgG mAb 390.

FIG. 9A through 9F provide evidence for the specific, inducible and enduring fibrinolytic activity of the anti-PECAM scFv-lmw scuPA fusion protein.

FIG. 9A is a line graph depicting amidolytic activities of the fusion protein, which is activated from its prodrug form by different molar ratios of plasmin to scFv-uPA. It demonstrates the concentration dependent activation of the prodrug to its active derivative.

FIG. 9B shows fibrinolytic activity using a fibrin plate. Equal doses (from left: 200, 100, 50 and 0 ng) of lmw-tcuPA (positive control, activated form of uPA), lmw-scuPA (positive control, prodrug form) and scFv-lmw scuPA fusion protein (in its prodrug form) were incubated on a fibrin-coated plate at 37° C. Lytic zones were measured after staining fibrin with Trypan blue.

FIG. 9C is a line graph depicting amidolytic activity associated with the cell surface of PECAM-negative REN (open circles) and PECAM-transfected (closed circles) REN cells determined by conversion of chromogenic substrate after incubation of the cells with various amounts of fusion proteins. The specific chromogenic substrate was added after washing of the cells.

FIG. 9D is a line graph showing that pre-incubation of REN/PECAM cells with the parental anti-PECAM IgG, mAb 390, reduces binding of enzymatically active scFv-uPA to the cells. This indicates that the binding and functional activity are antigen specific.

FIG. 9E is a bar graph showing the binding and amidolytic activity of the fusion protein after binding to murine cells at different time points.

FIG. 9F is a bar graph showing the binding and amidolytic activity of the fusion protein after binding to human cells at different time points. It demonstrates that the activity is preserved far longer on human cells than on murine cells.

FIG. 10A through 10C are bar graphs showing the biodistribution of anti-PECAM scFv-lmw scuPA and lmw-scuPA in vivo. For these experiments, 10 μg of fusion protein or lmw-scuPA were mixed with 0.25 μg of radiolabeled tracer protein and injected intravenously to wild type (WT) or PECAM null mice (PECAM KO), respectively. One hour later, tissue uptake was determined.

FIG. 10A is a bar graph showing the percentage of injected dose per gram tissue (% ID/g). scFv-uPA, but not scuPA, showed preferential uptake in the lungs and other highly vascularized organs in wild-type (WT), but not in PECAM Knockout (KO) mice.

FIG. 10B is a bar graph showing the organ-to-blood ratio for various organs. Broken line: blood level, ratio equal to 1. This confirms that the fusion protein is bound to the endothelium and that the increased uptake in highly vascularized organs (FIG. 10A) is not simply due to circulating protein in a higher volume of blood.

FIG. 10C is a bar graph showing the immunospecificity index (ISI), calculated as ratio of organ-to-blood ratios of targeted and untargeted counterpart. The broken line shows an ISI of 1, reflecting equal tissue levels of targeted and untargeted counterparts. Targeting to lung endothelium is approximately 10-fold higher with the targeted fusion protein.

FIG. 11A through 11C graphically depict the kinetics of in vivo pulmonary targeting and blood clearance of anti-PECAM scFv-scuPA.

FIG. 11A is a line graph comparing the kinetics of blood clearance of targeted fusion construct (closed circles) and non-targeted scuPA (open circles).

FIG. 11B is a line graph comparing the kinetics of the targeted fusion protein levels in lungs (closed circles) and blood (open circles). Fusion protein exhibited a rapid and prolonged accumulation in lung tissues. Lung-to-blood ratios at indicated time points were calculated and are shown in the inset.

FIG. 11C is a bar graph comparing the localization of the fusion protein scFv-uPA with untargeted uPA three hours after injection, demonstrating that the fusion protein is still present on the surface of pulmonary endothelium at this time point.

FIG. 12 is a dose-response curve of pulmonary thrombolysis. Dissolution of ¹²⁵I-labeled microemboli lodged in mouse pulmonary vasculature was measured following bolus injection of fusion protein (filled circles), the equivalent amount of lmw-scuPA (open circles) and a mixture of lmw-scuPA and parental antibody (filled triangles). Thrombolytic potency was expressed as percent lysis versus dose administered.

FIGS. 13A through 13E show the molecular design, migration, cleavage by thrombin and PECAM-1 binding of another exemplary fusion protein of the present invention, the anti-PECAM scFv-uPA pro-drug that is resistant to natural activation by plasmin, yet activated by thrombin, referred to herein as scFv/uPA-T fusion protein. FIG. 13A shows a construct of the single-chain Fv fused via linker SSSSGSSSSGAAA (SEQ ID NO:6) with thrombin-inducible lmw scuPA (scFv/uPA-T) generated by deleting Phe¹⁵⁷ and Lys¹⁵⁸ from the scFv/uPA construct. This leaves the sequence Pro¹⁵⁵-Arg¹⁵⁶-Ile¹⁵⁷-Ile¹⁵⁸ (SEQ ID NO:5), which is cleaved by thrombin after Arg¹⁵⁶. FIG. 13B is a photograph showing migration of the purified fusion protein in the absence or presence of thrombin analyzed using SDS-PAGE under unreduced or reduced conditions. FIG. 13C is a line graph depicting results from an ELISA binding anti-PECAM-scFv/uPA-T and free lmw-scuPA to immobilized mouse PECAM. FIG. 13D is a line graph depicting results from experiments measuring inhibition of binding of fusion protein to soluble mouse PECAM by parental anti-PECAM IgG. FIG. 13E is a line graph depicting results from experiments measuring thrombin-mediated release of the lmw-uPA moiety from PECAM-bound scFv/uPA. Purified scFv/uPA-T (25 μg/ml) was added to each well of a mouse PECAM-coated 96-well plate for 2 hour at 37° C. and washed with PBS. Thrombin (150 nM) was added for 2 hour at 37° C., the wells were washed and bound fusion protein was measured by ELISA. Error bars indicate standard error of the mean (s.e.m.).

FIG. 14A through 14F provide results from experiments demonstrating that thrombin, but not plasmin, induces the enzymatic activity of scFv/uPA-T. FIG. 14A and FIG. 14B are line graphs depicting amidolytic activity of scFv/uPA-T versus lmw-scuPA incubated with the indicated concentrations of thrombin or plasmin, respectively. FIG. 14C shows activity of scFv/uPA-T measured by zymography before and after exposure of the protein to thrombin analyzed under reduced or non-reduced condition. FIG. 14D shows fibrinolytic activity of indicated amounts of scFv/uPA-T, thrombin-treated scFv/uPA-T, lmw-tcuPA, lmw-scuPA and thrombin-treated lmw-scuPA incubated on a fibrin-coated plate at 37° C. Lytic zones were counterstained using impregnation of fibrin by Trypan blue. FIG. 14E shows amidolytic activity of scFv/uPA-T and lmw-scuPA bound to mouse PECAM after addition of thrombin. FIG. 14F shows susceptibility of scFv/uPA-T to PAI-1. Native scFv/uPA-T does not bind PAI-1. After addition of thrombin, a dose-dependent increase in scFv/uPA-PAI-1 complexes is evident.

FIG. 15A through 15D show results from experiments wherein scFv/uPA-T was administered intravenously to mice. FIG. 15A and FIG. 15B are bar graphs showing biodistribution of radiolabeled scFv/uPA-T versus wild type lmw-scuPA one hour (FIG. 15A) or three hours (FIG. 15B) after intravenous injection in mice. The data are shown as the percentage of the injected dose per gram of tissue (% ID/g), n=3, unless specified. FIG. 15C and FIG. 15D are bar graphs showing depletion of fibrinogen from mouse plasma in mice treated with wild-type lmw-scuPA, but not scFv/uPA-T. FIG. 15C shows the concentration of fibrinogen in plasma from mice treated with lmw-scuPA or scFv/uPA-T for 3 hours. FIG. 15D shows plasma fibrinogen levels of mice injected with 120 μg of scFv/uPA-T and the same dose of lmw-scuPA. scFv/uPA-T injected mice showed intact plasma fibrinogen level, in contrast to lmw-scuPA treated mice (n=6, P<0.05).

FIG. 16 is a bar graph depicting vascular-targeted scFv/uPA-T providing prophylactic fibrinolysis triggered by thrombin. Dissolution of pulmonary clots was provoked by thromboplastin injection in mice 0.5 hour or 3 hours after the bolus injection of equal amounts of scFv/uPA-T, scFv/uPA and lmw-scuPA. *, P<0.05, compared to lmw-scuPA; #, P<0.05, compared to scFv/uPA.

### DETAILED DESCRIPTION OF THE INVENTION

The present invention provides compositions and methods for use of these compositions in preventing coagulation, dissolving blood clots and protecting against intravascular thrombosis, either spontaneous or iatrogenic, as well as potential side effects following administration of a specific vascular occluding agent in cancer therapy and in treatment of other neoangiogenesis related diseases.

The compositions of the present invention comprise recombinant fusion proteins.

These fusion proteins comprise a ligand which specifically binds to endothelial surface determinants in a vascular bed of interest, thus providing the compositions of the present invention with the ability to act as thromboprophylactics in settings with high probability of intravascular thrombosis or embolism of circulating thrombi into pre-capillary vascular network such as the pulmonary or cerebral vasculature. Preferably the fusion protein binds monovalently to endothelial surface determinants, without cross-linking thereof. More preferably the ligand specifically binds to an endothelial cell surface molecule stably expressed or up-regulated in thrombosis and inflammation. Examples of such endothelial cell molecules include, but are not limited to, PECAM-1 and ICAM-1. These cell adhesion molecules are preferred because they poorly internalize monovalent conjugates such as the fusion proteins of the present invention and thus permit the composition of the present invention to reside for a relatively prolonged time on the luminal surface of endothelial cells.

An exemplary ligand for use in the fusion proteins of the present invention is an antibody to an endothelial cell molecule or a fragment of an antibody to an endothelial cell. Exemplary antibodies available through the ATCC include antibodies to PECAM-1 or ICAM-1 or an antigen-binding fragment of an antibody to PECAM-1 or ICAM-1. Examples of a fragment of an antibody useful in the fusion proteins of the present invention are monovalent antigen-binding domains such as scFv or Fab fragments of monoclonal antibodies directed against endothelial cell antigens such as PECAM-1 or ICAM-1.

The fusion proteins further comprise a fibrinolytic or thrombosis-inhibiting effector, also referred to herein as an anti-thrombotic molecule, linked to the ligand part of the construct via a linker peptide, thus forming a continuous polypeptide chain. The anti-thrombotic molecule has fibrinolytic, anticoagulant and/or platelet inhibiting activity.

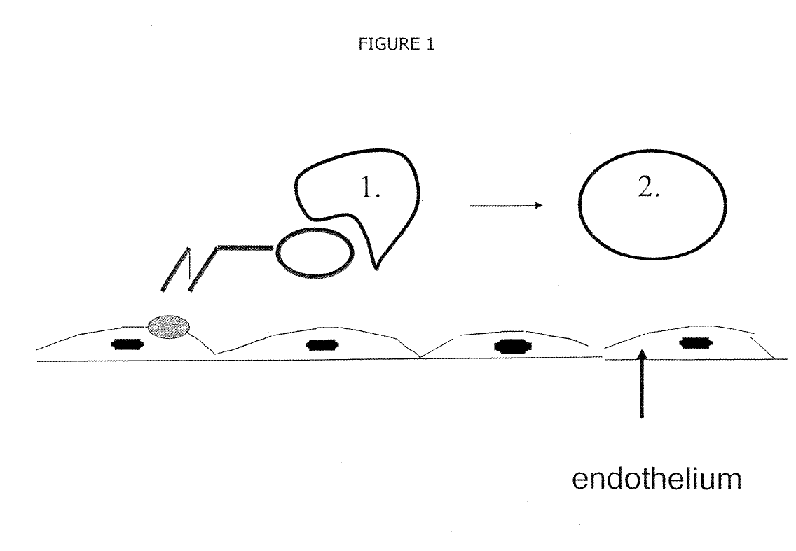

As depicted in FIG. 1, in one embodiment, the anti-thrombotic molecule is one which activates the precursor of a fibrinolytic compound or an anti-coagulant (substance 1 of FIG. 1) such as precursor of activated protein C or plasminogen to the active anti-coagulate (substance 2 of FIG. 1) such as activated protein C or plasmin. Examples of fibrinolytic or coagulation-inhibiting effectors useful in the present invention include, but are in no way limited to, plasminogen activators such as tissue plasminogen activator, urokinase, tenectase, retavase, streptokinase and staphylokinase or active fragments thereof and anticoagulants such as activated protein C, hirudin and agents inhibiting platelets.

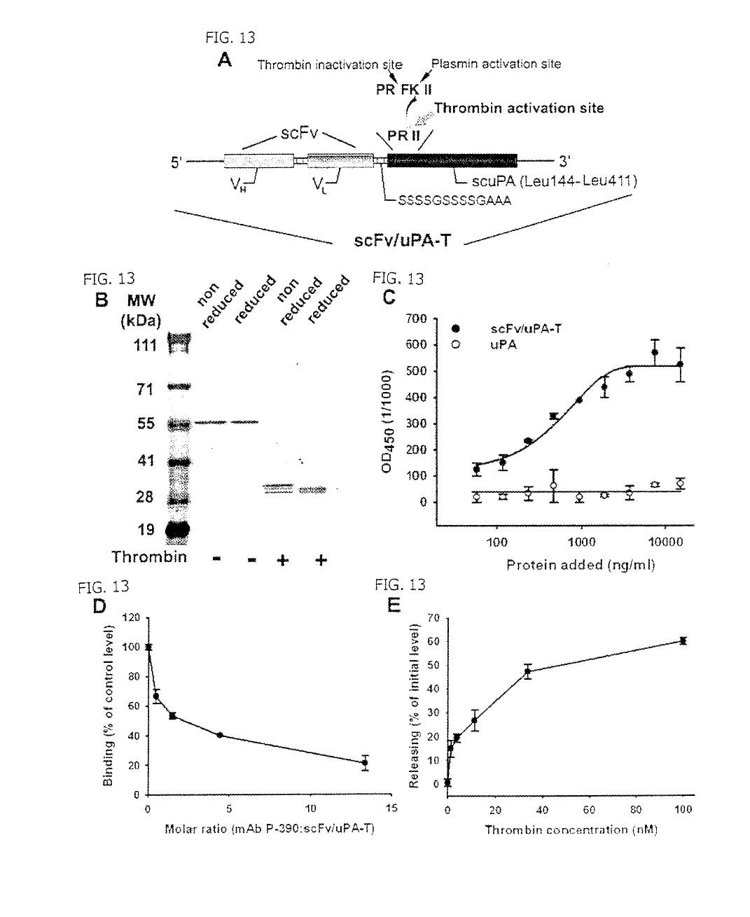

As depicted in FIG. 13A, in another embodiment, the fibrinolytic or coagulation inhibiting effector is modified to comprise an artificial thrombin activation site.

In addition, ligands that bind precursors of anticoagulants or fibrinolytics can be fused to the targeting moiety, for example thrombomodulin. An exemplary active fragment is the protease domain of any of the above-mentioned plasminogen activators.

Fusion proteins comprising an anti-thrombotic drug, preferably in a form of inactive pro-drug that is activated in the therapeutic site, with a monovalent scFv that binds to endothelial surface molecules without initiation of internalization characteristic of multimeric conjugates represent preferable agents for thromboprophylaxis. Molecules that are generated during the natural coagulation process and that can serve as activating molecules in this sense include but are not limited to fibrin, plasmin and thrombin. Such activating molecules are also referred to herein as pathological factors presented at the site of the pathological process and are not necessarily limited to those factors which are part of the coagulation system but may also include activation factors locally linked or connected to coagulation, for example inflammatory activation factors such as cytokines.

Furthermore, a potential limiting factor in all targeted therapeutic approaches is the impaired capability of the targeted molecules to diffuse into the clot. Since the therapeutic agent is bound to its target and therefore immobilized, diffusion capability is inhibited in all targeted approaches. Accordingly, in some embodiments of the fusion proteins of the present invention, a cleavage site is incorporated into the fusion protein between the binding moiety and the effector moiety, which is cleaved by a protease upon initiation of coagulation. Such cleavage sites may occur naturally or may be engineered at the respective site. Preferably the cleavage site is specific for a protease occurring during coagulation (including but not limited to serine proteases of the blood coagulation system). For example, it has now been found that the anti-PECAM scFv contains a natural short sequence of amino acids that form a thrombin-specific cleavage site, thus providing an ideal mechanism for local release and maximal activity of endothelium-bound drug in the site of active thrombosis. Accordingly in one embodiment, the fusion protein of the present invention comprises an anti-PECAM scFv portion possessing a natural thrombin cleavage site, providing site- and time-specific liberation of a drug in the site of active thrombosis.

The ligand which specifically binds to a vascular bed is linked to the anti-thrombotic molecule via a linker. Linkers of varying lengths have been used, e.g. 4 and 7 amino acids long. An exemplary linker of 13 amino acids is depicted in the scFV/uPA-T construct of FIG. 13A. Further it is expected that additional linkers ranging in length from as short as about 1 amino acid to as long as about 100 amino acids or longer can be used. In one embodiment a glycine3serine linker is used. However, as will be understood by the skilled artisan upon reading this disclosure, other linkers can be utilized in the present invention. In addition, a cleavage sequence, such as the thrombin-sensitive cleavage sequence can be inserted in the linker to provide for release of the drug upon active thrombosis.

In a preferred embodiment, the fusion proteins are produced recombinantly as a homogeneous substance. Such fusion proteins are capable of binding monovalently to endothelial surface determinants, without cross-linking thereof. Further, preferred is that the ligand and/or the fibrinolytic or coagulation inhibiting effector of the fusion protein be cleavable naturally or by insertion of an artificial cleavage site by thrombin or another specific protease that exists in an active form preferentially in the sites of active thromboses, thus providing a natural mechanism for local release of the drug in the site of its preferable action.

For example, in one embodiment, an additional thrombin cleavage site (Met-Tyr-Pro-Arg-Gly-Asn; SEQ ID NO:7) for plasminogen activator liberation can be appended to the linker between a ligand such as scFv and a fibrinolytic or coagulation inhibiting effector such as low molecular weight scuPA. For this embodiment, antibody-derived scFv with thrombin releasing site can be cloned by an upstream primer, which anneals to the amino terminus, and the downstream primer, which anneals to the carboxyl terminus and introduces the sequence including a short peptide linker with the thrombin cleavage site.

Several exemplary fusion proteins were generated in accordance with the present invention. One fusion protein comprises a scFv directed against murine PECAM and the kringle 2 and protease domain of tPA (scFv-tPA). Another one comprises the same scFv and low molecular weight single-chain pro-urokinase plasminogen activator (lmw-scuPA), termed scFv-uPA. Another comprises a latent single chain urokinase plasminogen activator (scuPA) coupled to a single chain antigen-binding fragment of PECAM-1 antibody (anti-PECAM scFv/scuPA) in which the endogenous plasmin activation site in low molecular weight scuPA is replaced with a thrombin-sensitive site and then fused to anti-PECAM scFv (scFv/scuPA-T).

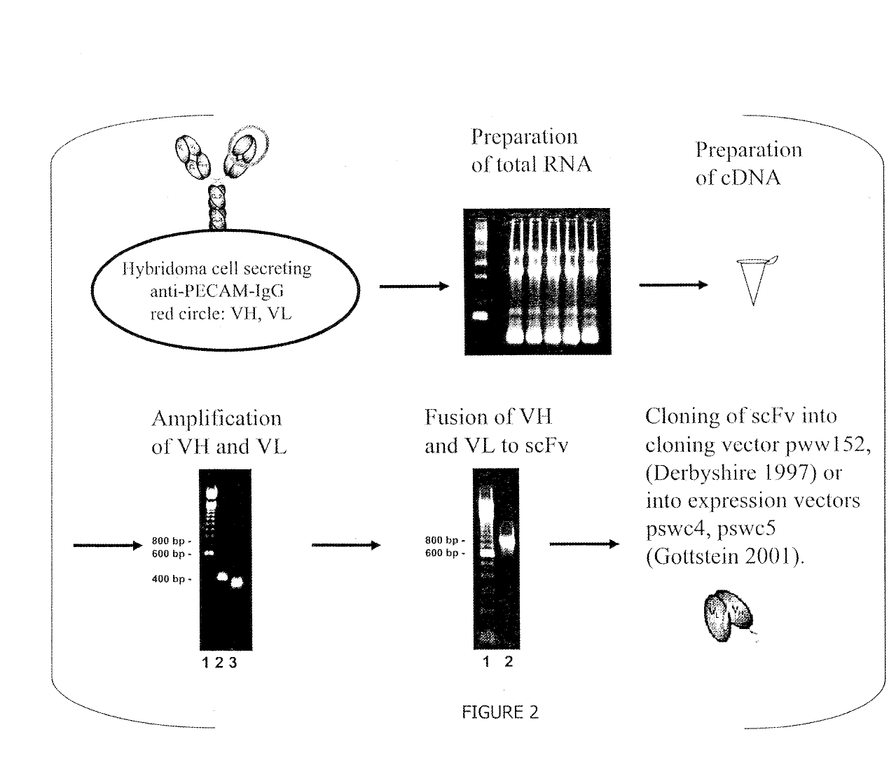

To generate these fusion proteins, anti-PECAM scFv was assembled from the hybridoma clone mAb 390 (Muzykantov et al. Proc Natl Acad Sci USA. 1999 96:2379-2384; Yan et al. J Biol. Chem. 1995; 270:23672-23680). RNA was extracted, cDNA from this RNA prepared and the cDNA encoding for the variable regions of the antibody amplified by PCR (polymerase chain reaction) as illustrated in FIG. 2 following the technology described by Derbyshire et al. (Immunochemistry 1: A practical approach. M. Turner, A. Johnston eds., Oxford University Press: 239-273, 1997). The variable regions of heavy and light chain (VH and VL) were fused to a synthetic scFv-antibody fragment using a (Gly₄Ser)₃ linker sequence. The scFv was cloned into plasmids, that allow easy reamplification for introduction into other vectors (from cloning vector pww152) or for expression as a scFv (from expression vector pswc5) or scFv tagged with soluble tissue factor (from expression vector pswc4). The plasmids pww152, pswc4 and pswc5 have been described in Derbyshire et al. (Immunochemistry 1: A practical approach. M. Turner, A. Johnston eds., Oxford University Press: 239-273, 1997) and in Gottstein et al. (Biotechniques 30: 190-200, 2001).

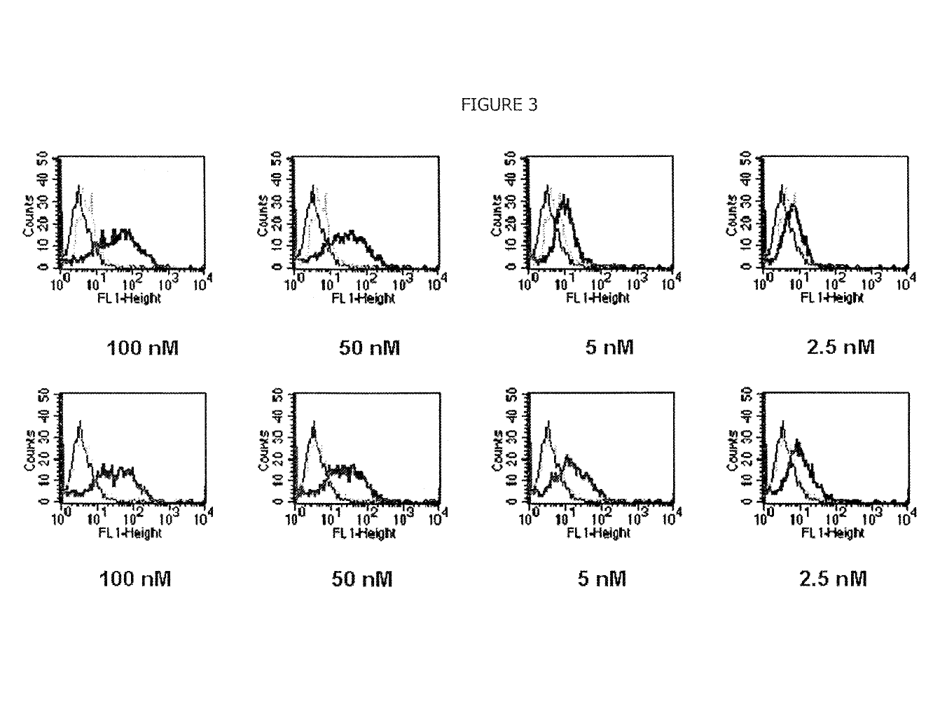

The binding of the scFv was confirmed by flow cytometry on PECAM positive murine endothelial cells (bEND3 after stimulation with tumor necrosis factor alpha). Binding was i) antigen specific, as evidenced by the lack of binding of appropriate negative controls, ii) dose dependant, iii) at a high affinity (50 nM) and iv) equal to the binding of the parental 390 IgG (FIG. 3).

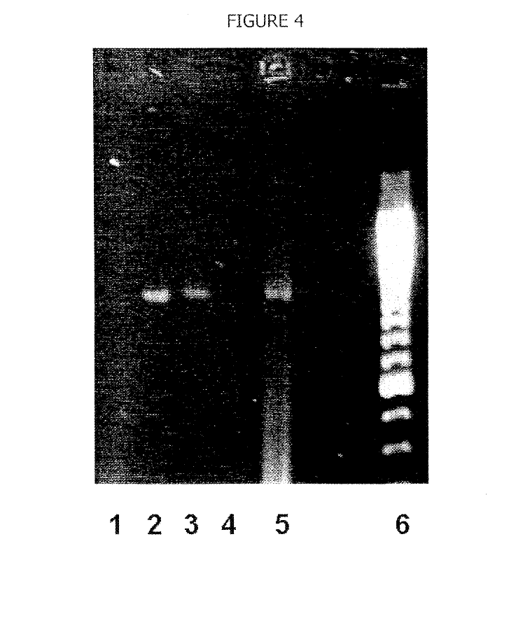

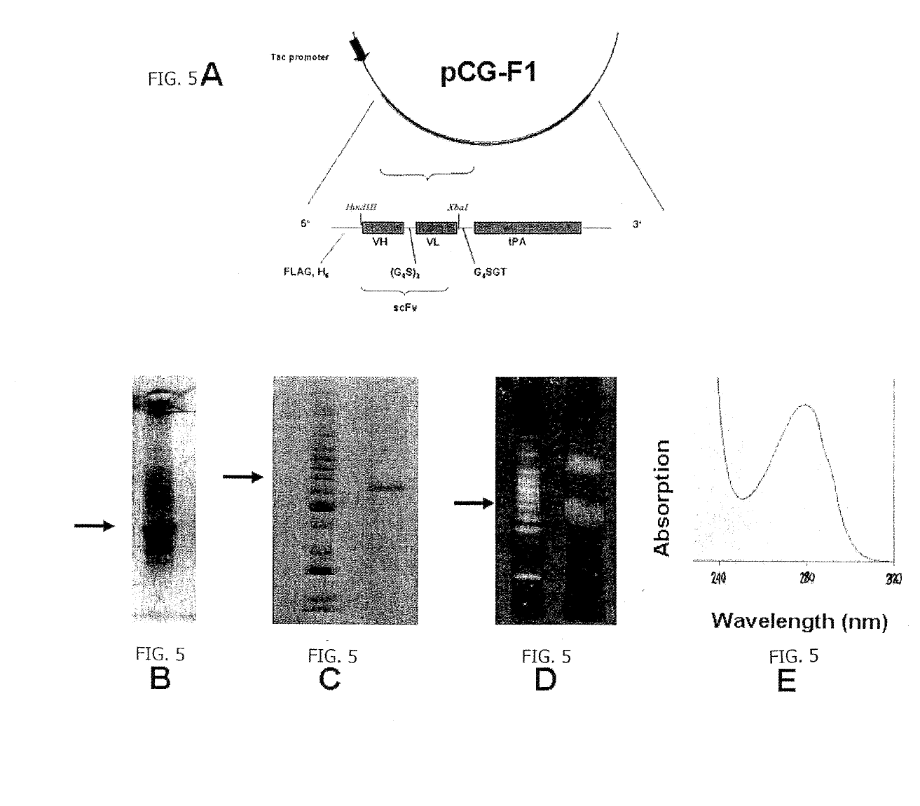

For the generation of the fusion protein scFv-tPA, the kringle 2 and protease domain (K2P) of tPA was cloned from human endothelial cells (FIG. 4), using the same technology of RNA-, cDNA- and DNA-preparation as illustrated in FIG. 2 and according to our published methods (Derbyshire et al. Immunochemistry 1: A practical approach. M. Turner, A. Johnston eds., Oxford University Press: 239-273, 1997). A new expression vector was prepared, by cloning the K2P domain of tPA into the plasmid pswc4. To this end, the soluble tissue factor DNA present in pswc4 (Gottstein et al. Biotechniques 30:190-200, 2001) was excised and replaced by the DNA fragment encoding the K2P domain of tPA. The resulting expression plasmid, pCG-F1 (FIG. 5A) contains a HindIII/XbaI cloning site upstream of the effector and downstream of tags used for detection and purification (FLAG, H6), allowing for easy unidirectional cloning of Hind III/XbaI-DNA-fragments. Using a modular set of cloning and expression vectors (Gottstein et al. Biotechniques 30:190-200, 2001), scFv-fragments containing the appropriate restriction sites such as HindIII and XbaI allow for rapid exchange of the targeting moiety.

The fusion protein was then expressed in E. coli and purified to homogeneity, as evidenced by Coomassie Blue and Silver stained SDS-PAGE (FIGS. 5C and D). The identity of the protein was confirmed by western blotting using an antibody against human tPA as a detection antibody (FIG. 5A). Scanning spectrophotometry showed a typical protein profile (peak at 278 nm, shoulder after 280 nm) without contaminants (FIG. 5E).

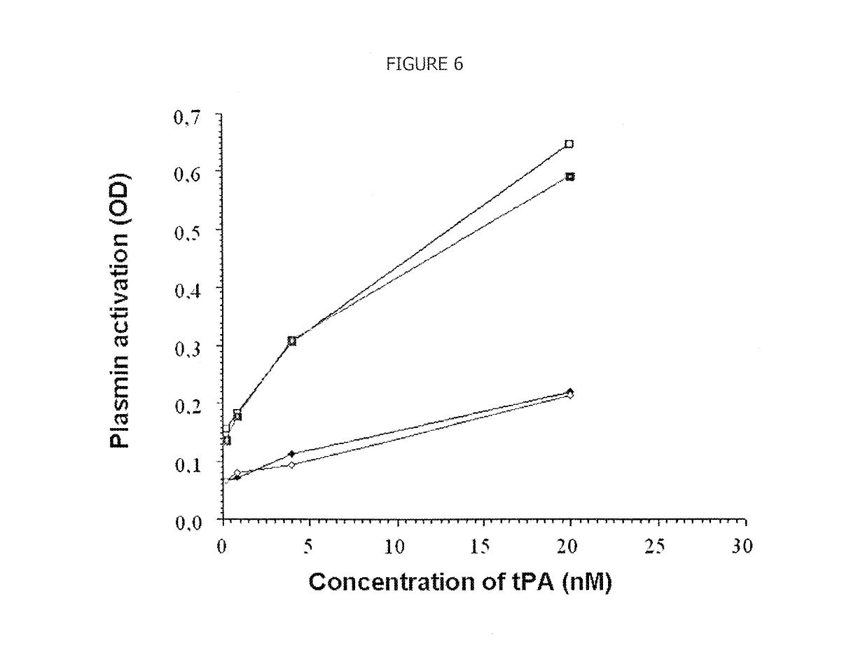

Functional analysis of scvFv-tPA revealed, that it was 1) fibrinolytically active, since it activated plasminogen to plasmin in a dose-dependent manner and 2) and fibrin-specific since its activity is enhanced in the presence of fibrin, i.e. when clotting occurs (see FIG. 6).

These data therefore show that the K2P domain of tPA can be linked to a scFv targeting moiety without loss of the fibrinolytic activity and without loss of its fibrin specificity. The latter feature offers a naturally designed prototype for local augmentation of the fibrinolytic activity of the endothelium-anchored fusion construct by a local pathological factor formed in thrombosis, namely fibrin.

The second fusion protein, scFv-uPA, was also designed to gain full fibrinolytic activity in the presence of coagulation and/or fibrinolysis processes, but in a different manner. In this exemplary embodiment, cleavage of this scFv/lmw-scuPA pro-drug by plasmin generated fibrinolytically active two-chain lmw-uPA. This fusion protein: i) bound specifically to PECAM-1 expressing cells; ii) was rapidly cleared from blood after intravenous injection; iii) accumulated in the lungs of wild-type C57BL6/J, but not PECAM-1 null mice; and, iv) lysed pulmonary emboli formed subsequently more effectively than lmw-scuPA, thereby providing proof of principle of thromboprophylaxis using recombinant scFv-fibrinolytic fusion proteins that target endothelium.

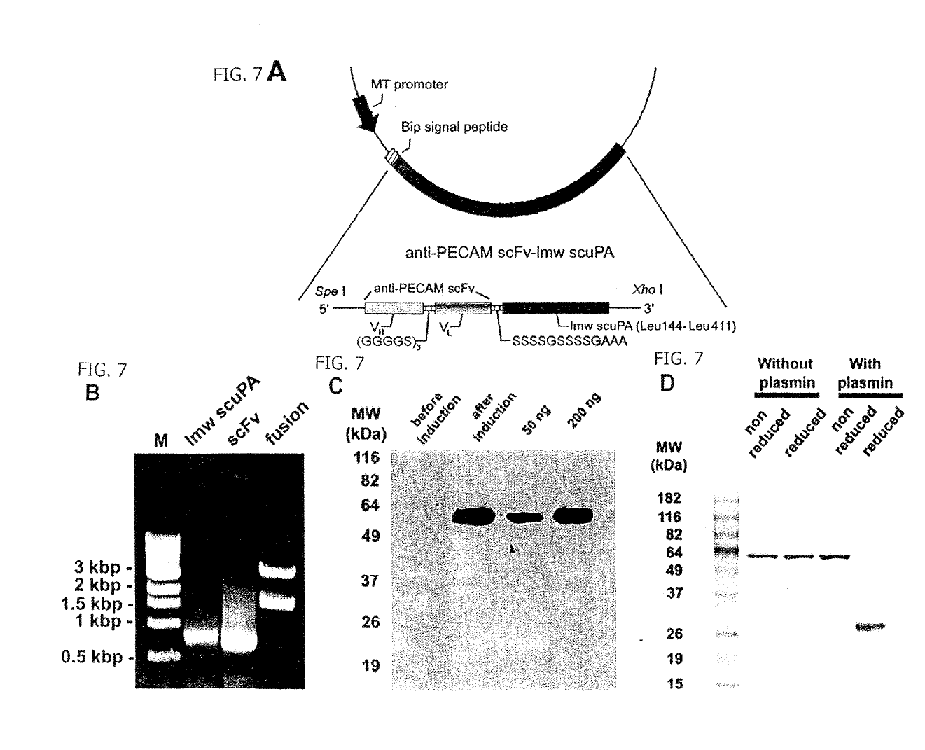

In these experiments, DNA encoding scFv was fused with DNA encoding lmw-scuPA using a (Ser₄Gly)₂Ala₃ linker, yielding the plasmid pMT-BD1, which encodes for the fusion protein scFv/lmw-scuPA (scFv-uPA, unless specified otherwise) (FIG. 7B). scFv-uPA expression was induced in S2 drosophila cells as previously described (Bdeir et al. Blood. 2003 102:3600-3608), and the fusion protein was purified from cell media with a yield of 5 mg/L.

The protein migrated as a single band at the predicted size (˜60 kDa) on SDS-PAGE under reducing conditions and its identity was confirmed by western blotting using an anti-uPA antibody (FIG. 7C). The fusion protein was cleaved by plasmin into a two-chain derivative (lmw-tcuPA) composed of two nearly identically-sized fragments, the N-terminal portion of the fusion protein comprising scFv linked to amino acids Leu¹⁴⁴-Lys¹⁵⁸ of uPA (30 kDa) and the B-chain of uPA (amino acids Ile¹⁵⁹-Leu⁴¹¹, MW 30 kDa), which co-migrate and therefore appear as a single band (FIG. 7D).

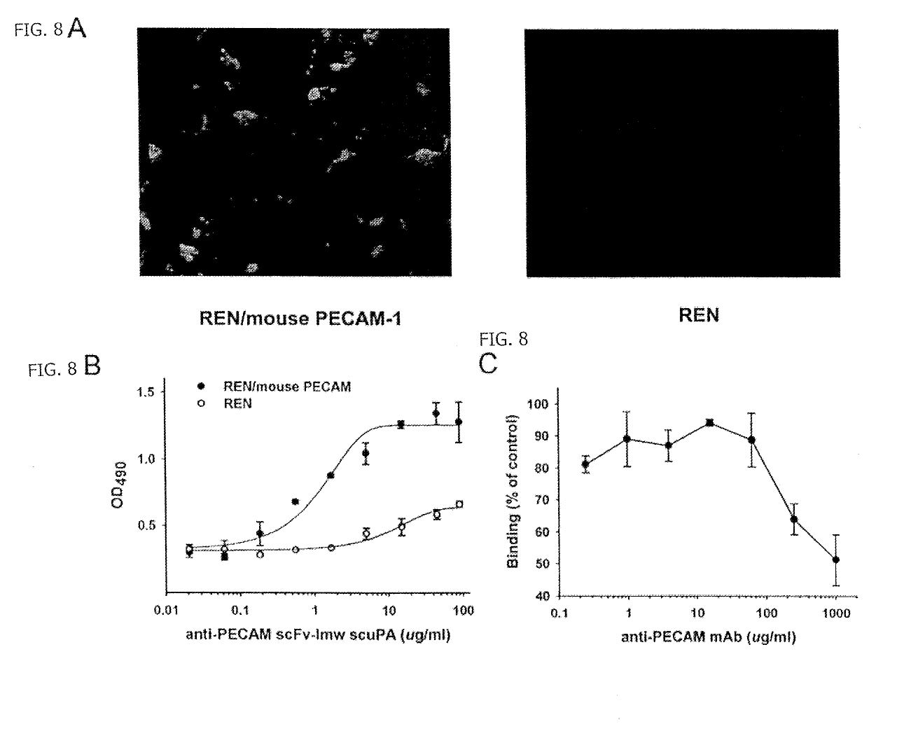

Binding of scFv-uPA to PECAM-expressing cells was examined Biotinylated scFv-uPA bound to a human mesothelioma cell line REN (Vaporciyan et al. Science. 1993 262:1580-1582) transfected with cDNA encoding murine PECAM-1 (REN/PECAM), but not to untransfected control REN cells assessed using FITC-labeled streptavidin and immunofluorescence microscopy (FIG. 8A). Specific binding of scFv-uPA to PECAM-expressing cells was confirmed by ELISA (half-maximal binding 38 nM; FIG. 8B). Addition of free anti-PECAM IgG inhibited scFv-uPA binding to REN/PECAM cells, confirming the specificity of targeting (FIG. 8C).

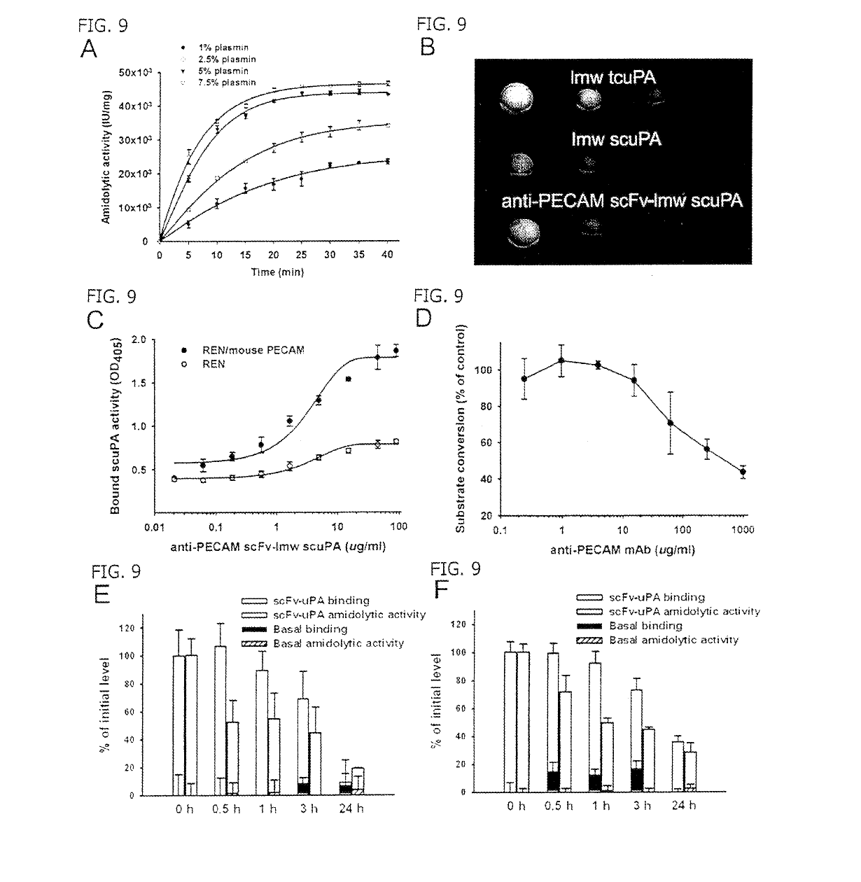

Enzymatic and fibrinolytic activity of anti-PECAM scFv-uPA was assessed. Plasmin converted scFv-uPA fusion protein into two-chain lmw-urokinase (FIG. 7D). To verify that this produces an active urokinase from a latent pro-drug, its enzymatic activity was tested using a chromogenic substrate. Plasmin induced a marked, dose-dependent increase in the amidolytic activity of scFv-uPA, from a basal level of ≦5,040 IU/mg to 46,590 IU/mg (FIG. 9A). scFv-uPA lysed preformed fibrin clots containing plasminogen to the same extent as free lmw-scuPA (FIG. 9B). Therefore, N-terminal fusion of lmw-uPA to the scFv did not compromise its folding, ability to become activated by plasmin, or its ability to initiate fibrinolysis.

REN/PECAM cells incubated with scFv-uPA developed cell surface enzymatic activity whereas control REN cells incubated in the same way did not (FIG. 4C). Addition of the parental anti-PECAM IgG inhibited the delivery of uPA activity to target cells, confirming the specificity of targeting (FIG. 9D).

The binding of scFv-uPA to the surface of mouse endothelial cells was relatively stable as determined by ELISA. The half-life of bound scFv-uPA was approximately 5 hours, but the protein was not detected at 24 hours. The enzymatic activity of cell-bound scFv-uPA declined by 50% within 30 minutes, but was sustained thereafter at this level for approximately 3 hours (FIG. 9E). The longevity of enzymatically active scFv-uPA anchored to the surface of human REN/PECAM cells was even more prolonged: approximately 40% of initial levels of bound scFv-uPA antigen and its activity remained on the cell surface 24 hours after binding (FIG. 9F).

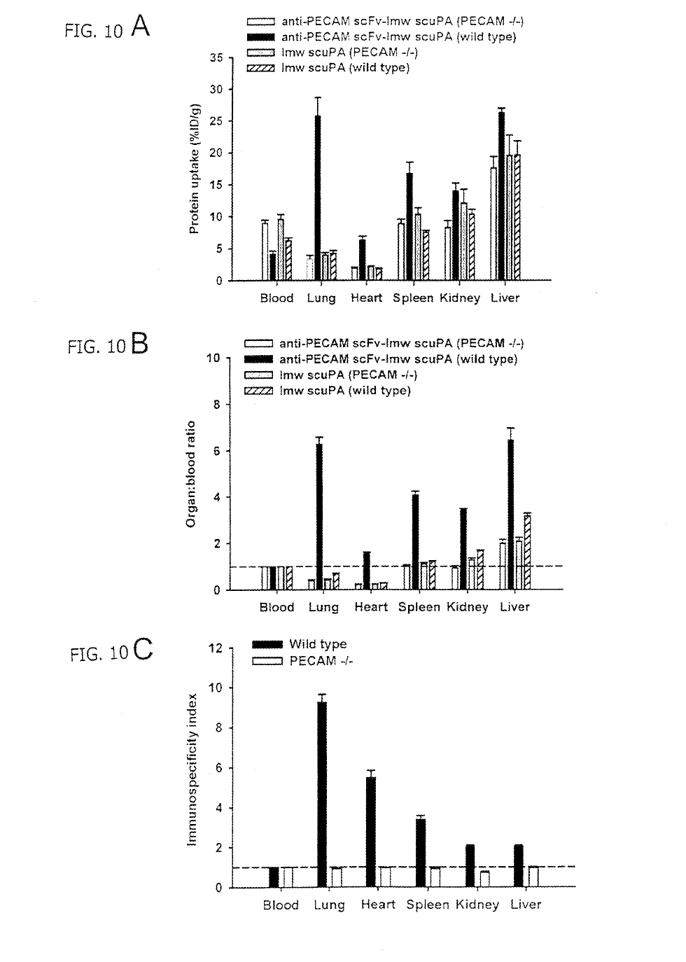

Vascular immunotargeting of scFv-uPA in mice was also examined To test the blood clearance, biodistribution and endothelial targeting of scFv-uPA and control formulations, each was radiolabeled with ¹²⁵I and injected into mice. One hour after intravenous injection, free ¹²⁵I-lmw-scuPA was distributed similarly in the blood of wild type and PECAM KO mice (FIG. 10A). Notably, the organ-to-blood ratio, a parameter that reflects preferential uptake in organs of interest, did not exceed 1 in any organ, except for the liver, which serves as a site of clearance of plasminogen activators from blood. This result shows that free lmw-scuPA injected in circulation does not show significant binding to endothelial cells (FIG. 10B).

The organ distribution of ¹²⁵I-labeled scFv-uPA in PECAM KO mice was nearly identical to that of non-targeted lmw-scuPA (FIG. 10A). In contrast, the fusion protein accumulated preferentially in the lungs and, to somewhat lesser extent, in other highly vascularized organs of wild type mice expressing PECAM on the surface of endothelium (FIG. 10A). The scFv-uPA immunospecificity index (ISI, ratio of tissue uptake of targeted versus non-targeted counterparts, a marker of targeting specificity) was 10 in the lungs and 5 in the heart of wild type mice (FIG. 10C). In contrast, the ISI of scFv-uPA did not exceed 1 in any organ in PECAM null mice, thus demonstrating that no specific uptake occurred in the absence of endothelial targeting.

Anti-PECAM scFv-uPA was cleared from the circulation more rapidly than non-targeted lmw-scuPA (FIG. 10A, 11A), suggesting depletion of the circulating pool as a result of endothelial binding of the fusion protein. In agreement with this interpretation, the blood levels of scFv-uPA and scuPA were identical in PECAM KO mice (FIG. 10A).

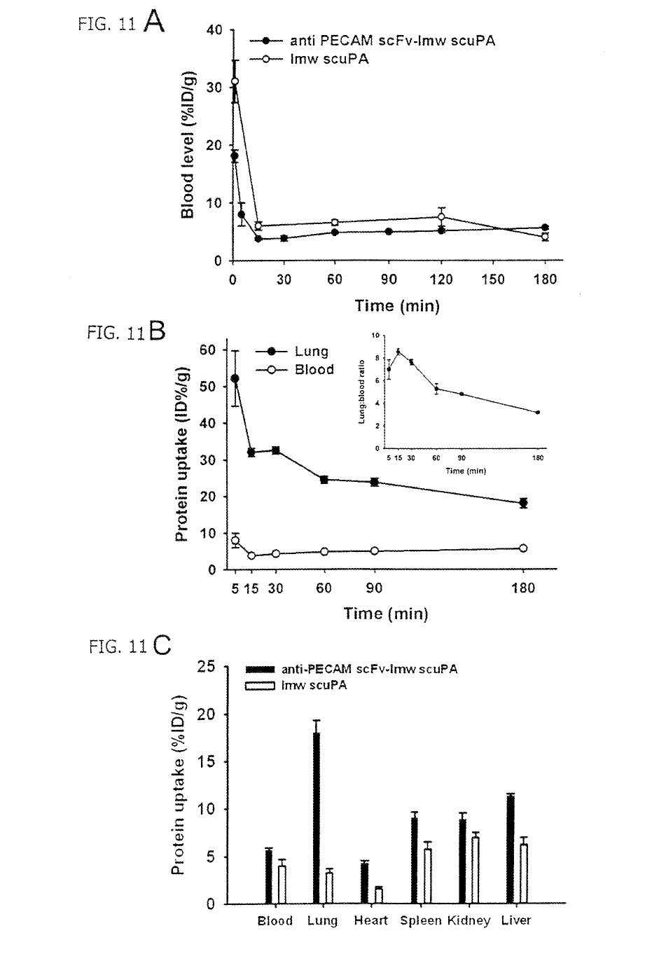

Uptake of the fusion protein in the lung of wild-type (WT) mice was rapid, reaching its maximum 5 minutes post injection (FIG. 11B). After an initial 30% decline due to blood clearance, the lung/blood ratio of scFv-uPA peaked at 15 to 30 minutes and was relatively stable over the next several hours.

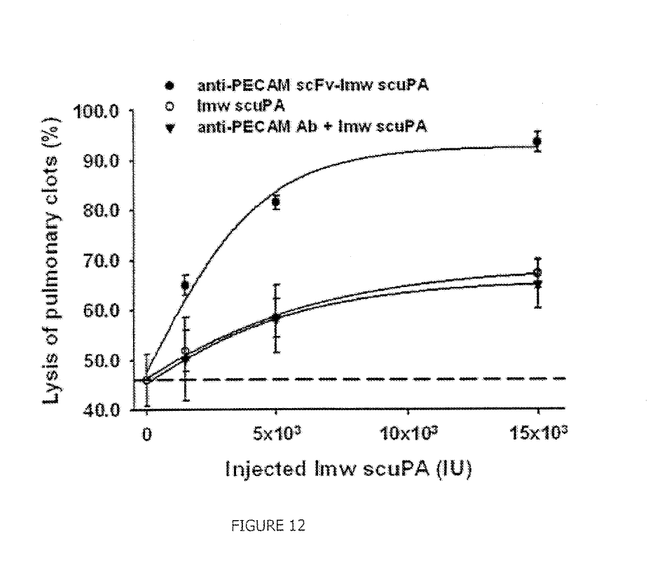

Given these favorable targeting and kinetic characteristics, the effect of scFv-uPA delivery to endothelial PECAM in a mouse model of acute pulmonary thrombosis induced by injecting radiolabeled fibrin emboli (3-5 μm in diameter) was examined. After intravenous injection these emboli form aggregates that lodge preferentially in the pre-capillary bed of the lungs (Murciano et al. Am J Physiol Lung Cell Mol. Physiol. 2002 282:L529-L539). To model prophylactic fibrinolysis, various doses of scFv-uPA and the same amount of non-targeted lmw-scuPA were injected prior to injecting ¹²⁵I-emboli. The residual isotope in the lungs was measured 1 hour later. At all doses tested, the fusion protein produced significantly greater clot lysis than enzymatically identical doses of non-targeted lmw-scuPA (P<0.025) (FIG. 12). This effect could not be attributed to the potential benefit of blocking of PECAM-1, since fibrinolysis by a mixture of lmw-scuPA and anti-PECAM did not exceed that produced by lmw-scuPA alone.

The third exemplary fusion protein, scFv/scuPA-T, in which the endogenous plasmin activation site in low molecular weight scuPA was replaced with a thrombin-sensitive site and then fused to anti-PECAM scFv was designed to provide more highly localized and durable thromboproprophylaxis. scFv/scuPA-T: i) bound specifically to mouse PECAM-1; ii) accumulated preferentially in mouse lungs after IV injection; iii) was inactive and resistant to plasma inhibitor PAI-1 until converted to active two-chain urokinase and released by thrombin; and, iv) did not consume fibrinogen from plasma after IV injection. In a mouse model of thrombin-mediated thrombosis, scFv/scuPA-T, but not wild-type lmw-scuPA, mediated/enhanced pulmonary fibrinolysis. Moreover, scFv/uPA-T showed a significantly enhanced in vivo durability, compared to plasmin-sensitive scFv/scuPA. Fusion proteins of the present invention designed in accordance with this exemplary embodiment provide a means for endothelial-targeted thromboprophylaxis triggered by pro-thrombotic enzymes and a general approach towards regulating cell-associated proteolytic reactions in a time- and site-specific manner.

This exemplary fusion protein scFv/uPA-T of the present invention was constructed using cDNA encoding lmw scuPA fused to anti-PECAM scFv as a template. The plasmin cleavage site Phe¹⁵⁷-Lys¹⁵⁸ was deleted thereby creating the thrombin-cleavage site Pro¹⁵⁵-Arg¹⁵⁶-Ile¹⁵⁷-Ile¹⁵⁸ (SEQ ID NO:5; FIG. 13A). scFv/uPA-T protein migrated on SDS-PAGE as a single band at the predicted molecular weight (˜59 kDa) under reduced conditions, excluding activation in vitro (FIG. 13B). Anti-PECAM scFv contains one potential thrombin cleavage site (Pro²³²-Arg²³³-Ala²³⁴; SEQ ID NO:8) predicted to yield protein fragments of ˜30 kD. Thus, thrombin cleaved the fusion protein within scFv, generating two proteins with distinct migrations under non-reduced conditions (FIG. 13B). Under reduced conditions, the B-chain dissociated from thrombin-cleaved scFv/uPA-T leading to faster migration (FIG. 13B).

scFv/uPA-T, but not non-targeted lmw-scuPA, bound to the immobilized extracellular domain of mouse PECAM (FIG. 13C). Binding was inhibited by a PECAM monoclonal antibody (FIG. 13D). Thrombin released uPA-T from PECAM-bound scFv/uPA-T (FIG. 13E), implying that thrombin might activate and liberate lmw-uPA at sites of thrombosis in vivo. In these experiments, purified scFv/uPA-T (25 μg/ml) was added to each well of a mouse PECAM-coated 96-well plate, incubated for 2 hour at 37° C., and washed with PBS. Thrombin (150 nM) was added for 2 hours at 37° C., the wells were washed and bound fusion protein was measured by ELISA.

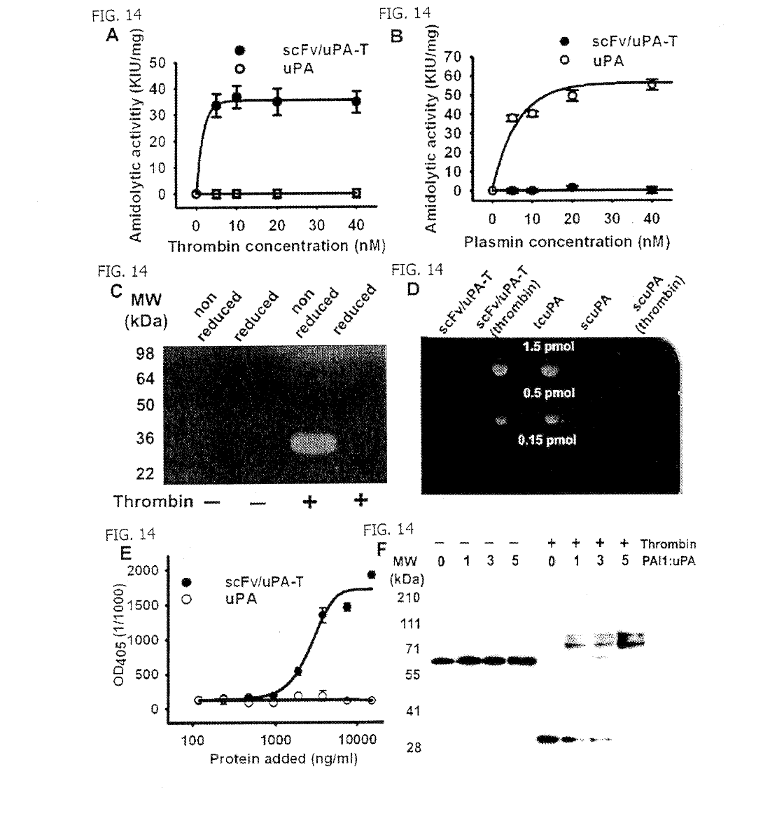

In fact, cleavage of scFv/uPA-T by thrombin generated amidolytic activity while cleavage of lmw-scuPA does not (FIG. 14A); conversely, plasmin cleaved lmw-scuPA but not scFv/uPA-T (FIG. 14B). scFv/uPA-T does not express plasminogen activator activity on zymography. Cleavage of scFv/uPA-T by thrombin generated a approximately 30 kD fragment that activated plasminogen and that lost this activity under reduced conditions, consistent with the expected requirement for disulfide-bonding (FIG. 14C). scFv/uPA-T did not lyse fibrin clots containing trace amounts of plasminogen, whereas thrombin-activated scFv/uPA-T expressed fibrinolytic activity (FIG. 14D, left columns) comparable to plasmin-generated lmw-tcuPA used as a positive control (FIG. 14D, central column). The low intrinsic PA activity of lmw-scuPA was eliminated by thrombin, as expected (FIG. 14D, right columns). Adding thrombin to PECAM-coated plastic wells pre-incubated with scFv/uPA-T, but not lmw-scuPA, generated amidolytic activity, indicating that antigen-bound scFv/uPA-T maintains its susceptibility to thrombin activation (FIG. 14E). Native scFv/uPA-T did not bind PAI-1 even at 5-fold molar excess inhibitor (FIG. 14F, left lanes). However, thrombin generated an approximately 30 kD two-chain uPA fragment of scFv/uPA-T that expressed enzymatic activity (FIG. 14C) and formed SDS-resistant complexes (Manchanda, N. and Schwartz, B. S. J Biol Chem 1995 270:20032-20035) with the inhibitor (FIG. 14F, right lanes).

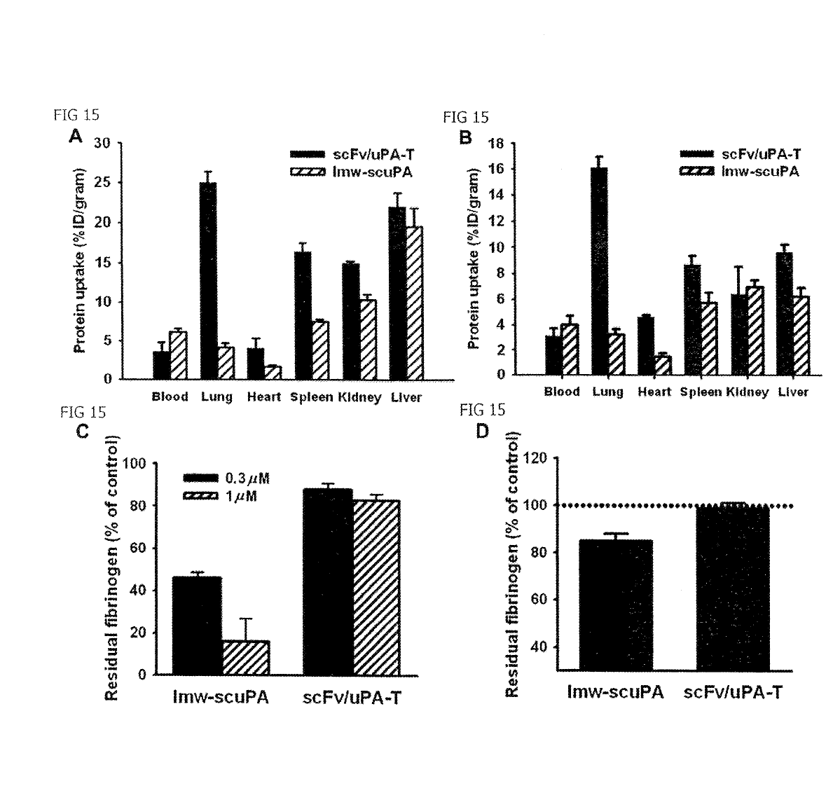

Radiolabeled scFv/uPA-T accumulated in the lungs after IV injection in mice, whereas lmw-scuPA did not (FIG. 15A). This result was consistent with previous disclosures that PECAM-targeted drugs accumulate in the pulmonary vasculature due to binding to endothelial PECAM in this highly vascularized organ (Muzykantov et al. Proc Natl Acad Sci USA 1999 96:2379-2384; Christofidou-Solomidou et al. Am J Physiol Lung Cell Mol Physiol 2003 285:L283-292; Kozower et al. Nat Biotechnol 2003 21:392-398). Both scFv/uPA-T and wild-type lmw-scuPA were cleared rapidly from the circulation (4% and 6% ID/g blood at 30 minutes post injection). However, the blood level of scFv/uPA-T was even lower, consistent with binding to endothelial PECAM. Even 3 hours after injection, the amount of scFv/uPA-T in the lungs was approximately 5 fold higher than that of non-targeted uPA (FIG. 15B), whereas the blood level of scFv/uPA-T remained lower than lmw-scuPA throughout.

Experiments were then performed to examine whether scFv/uPA-T generated less indiscriminate systemic activity as a result of its more rapid blood clearance and lack of constitutive activity. To exclude pharmacokinetic factors, fibrinogen consumption was assessed in vitro. lmw-scuPA (0.3 and 1 μM) depleted fibrinogen from mouse plasma (approximately 50 and 20% of normal, respectively), whereas fibrinogen levels remained above 80% after incubation of plasma with scFv/uPA-T (FIG. 15C). The same disparity was seen in vivo. One hour after injection of 120 μg (2 nmoles) of scFv/uPA-T, the concentration of fibrinogen in mouse blood was unaffected, while the same molar amount of lmw-scuPA caused 15% depletion (P<0.05) (FIG. 15D).

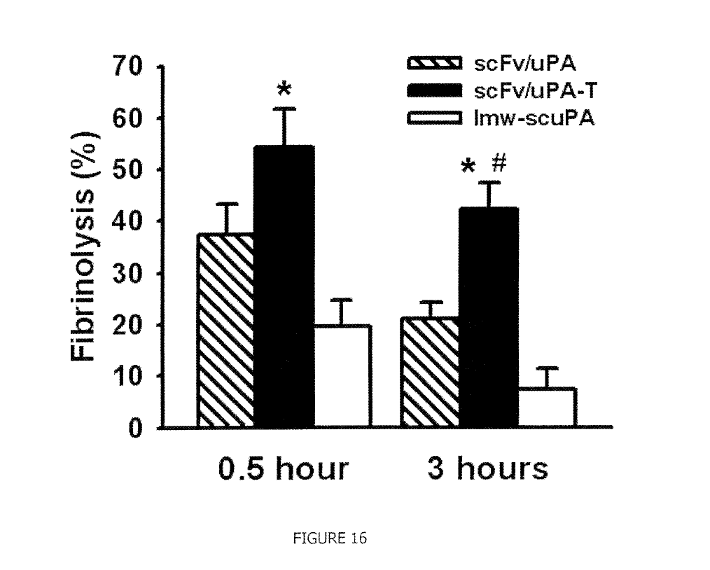

To test prophylactic thrombolytic activity in vivo, scFv/uPA-T or lmw-scuPA was intravenously injected into mice prior to the injection of a mixture of thromboplastin and ¹²⁵I-fibrinogen. The residual radioactivity in lungs was measured 90 minutes later to monitor deposition and lysis of thrombi that form intravascularly. In agreement with previous reports (Leon et al. Circulation 2001 103:718-723; Smyth et al. Blood 2001 98:1055-1062), injection of thromboplastin lead to the pulmonary fibrin deposition in control mice. Prophylactic injection of lmw-scuPA, 0.5 or 3 hours before thromboplastin, had a non-significant effect on fibrin deposition (P>0.3) (FIG. 16). While the fibrinolysis induced by plasmin-sensitive scFv/uPA decreased during 3 hours, scFv/uPA-T injected three hours prior to thromboplastin significantly augmented clot lysis in the pulmonary vasculature (P<0.05) (FIG. 17).

Thus, as shown by these experiments, these exemplary recombinant fusion proteins of the present invention target a fibrinolytic pro-drug to a luminal endothelial cell antigen. In fact, these fusion proteins specifically target endothelial cells in vitro and in vivo and provide antigen-specific enhancement of fibrinolytic activity in a mouse model of pulmonary thrombosis, thus indicating that vascular immunotargeting with these fusion proteins can be utilized for prophylactic and therapeutic fibrinolysis.

Accordingly, compositions of the present invention comprising these fusion proteins can be administered to an animal, preferably a human, as a prophylactic to prevent clot formation. The modular design of the fusion proteins allows for easy replacement of targeting moieties with scFv-antibodies directed against endothelial targets in a desired species, e.g. humans. As illustrated in FIGS. 9E and F, the effect in humans is probably underestimated when extrapolating the results from mice, because the fusion protein scFv-uPA remained active longer on human cells, and because it is well known, that murine inactivators of urokinase are very effective in inhibiting human urokinase.

When used prophylactically, it is preferred that the animal first be identified as having a high propensity of intravascular thrombosis or thromboembolism, either recurrent due to an existing pathological condition or nascent due to high risk of intravascular thrombosis associated with medical interventions. Examples of pathological conditions associated with an animal having a high propensity for recurrent thrombosis or thromboembolism include, but are not limited to, pulmonary embolism, myocardial infarction, stroke, deep vein thrombosis and thrombosis associated with patient immobilization, advanced age, transient ischemic attack, prior venous thromboembolic disease, cancer patients treated with hormonal therapy, chemotherapy or radiotherapy, acute medical illness, respiratory failure, inflammatory bowel disease, nephrotic syndrome, varicose veins, central venous catheterization, and inherited or acquired thrombophilia. In these settings, binding of active anti-thrombotic agents to the endothelial luminal surface will inhibit formation or facilitate dissolution of secondary blood clots. Examples of nascent clotting due to high risk of intravascular thrombosis associated with medical interventions, which can be predicted with high probability include, but are not limited to, clots formed in the pulmonary vessels of patients undergoing mechanical ventilation and hyperoxia, sickle cell anemia patients undergoing blood transfusions, as well as patients recovering after surgical interventions, treated with estrogens or agents which provoke thrombosis in tumor vasculature. In these settings, binding of active anti-thrombotic agents to endothelial luminal surface will inhibit formation or facilitate dissolution of primary nascent blood clots.

Further, the ability to introduce an artificial protease cleavage site such as a thrombin cleavage site into the fusion proteins of the present invention or to select a ligand with a natural protease cleavage site for use in the fusion proteins of the present invention provides for selective therapeutic composition activated locally at a site of a pathological process such as a site of active thrombosis by a pathological factor such as thrombin presented at the site. Such compositions are useful in methods of promoting local release of an anti-thrombotic agent at a site of active thrombosis in an animal.

The compositions of the present invention are especially useful in protecting against potential unwanted side effects following administration of a therapeutic or diagnostic agent or procedure that potentially increases the risk for thrombosis such as specific vascular occluding agents or other cancer treatment agents in cancer therapy and other neoangiogenesis related diseases. In this embodiment, a composition comprising a fusion protein of the present invention is coadministered to an animal with the specific vascular occluding agent.

By co-administered as used herein, it is meant that the fusion protein is administered prior to, at the same time, or after the specific vascular occluding agent or cancer treatment agent.

In addition to the fusion protein, compositions of the present invention may further comprise a pharmaceutically acceptable vehicle for intravenous administration or administration via other vascular routes including but not limited to intra-arterial and intra-ventricular administration, as well as routes providing slower delivery of drugs to the bloodstream such as intramuscular administration to an animal in need thereof or at risk for uncontrolled intravascular fibrin clot formation. Examples of such pharmaceutically acceptable vehicles include, but are not limited to, saline, phosphate buffered saline, or other liquid sterile vehicles accepted for intravenous injections in clinical practice. In one embodiment, compositions of the present invention are administered systemically as a bolus intravenous injection of a single therapeutic dose of the fibrinolytic or coagulation-inhibiting effector (for example, 0.1-1.0 mg/kg for plasminogen activators). However, as will be understood by those of skill in the art upon reading this disclosure, alternative dosing regimes and modes of administration may be used depending upon the age, weight and condition of the animal being treated.

Fusion proteins of the present invention can be administered via diverse routes, each optimally serving certain medical needs. For example, systemic intravascular route can be used to augment anti-thrombotic potential in the entire vasculature. This type of intervention would be helpful in many thrombotic settings which in general affect many vascular beds. In particular, intravenous injection provides preferential accumulation of these fusion proteins in the pulmonary vasculature, a very often target for thrombosis and thromboembolism. Injection in an artery feeding a given organ (e.g., via vascular catheter) will provide enriched accumulation and subsequent anti-thrombotic effect in an organ of specific interest, such as the heart (delivery via coronary artery) for prophylaxis of AMI and unstable angina, or the brain (via cerebral artery), for prophylaxis of stroke and other cerebrovascular thrombotic events. Further, these fusion proteins can be administered into an organ donor, or into a perfusion solution in the isolated organ transplant, prior to transplantation into a recipient patient, for prophylaxis of post-ischemic thrombosis and complications of the graft.

By “animal” as used herein it is meant to be inclusive of any mammal including humans.

The following nonlimiting examples are provided to further illustrate the present invention.

### EXAMPLES

### Example 1

### Generation and Binding Analysis of Anti-CD31-scFv

scFvs directed against luminal endothelial cell antigens were generated in accordance with teachings herein and teachings of Gottstein et al. (Biotechniques 2001 30:190-200). An example is an anti-CD31-scFv, derived from the hybridoma cell line 390. The corresponding antibody, Mab 390, is a rat monoclonal antibody directed against murine PECAM-1. The variable regions of the P-390 antibody heavy and light chains were cloned into the plasmid pww152 essentially as described previously by Derbyshire et al. (Immunochemistry 1: A practical approach. M. Turner, A. Johnston eds., Oxford University Press: 239-273, 1997). The variable heavy chain and light chain were assembled into a scFv fragment by overlap extension PCR and cloned into the expression plasmid pswc4 (Gottstein et al. Biotechniques 2001 30:190-200).

The scFv-protein was expressed with a tag (soluble tissue factor), which allows easy detection in binding assays, and which has no background binding activity to endothelial cells. Proteins were expressed in E. coli and purified by affinity chromatography (Gottstein et al. Biotechniques 2001 30:190-200). Binding of the recombinant scFv antibody was tested by flow cytometry in comparison with the parental IgG antibody. The scFv bound with a nanomolar affinity (half-maximal binding about 50 nM), and showed identical binding characteristics on CD31 positive endothelial cells (bEND3, from B. Engelhard, Max-Planck Institute, Bad Nauheim, Germany) as the parental IgG (FIG. 3).

### Example 2

### Production and Biochemical Characterization of scFv-Anti-CD31-tPA Fusion Protein

The DNAs of a scFv against CD31 and a K2P (kringle 2 protease) domain of human tissue plasminogen activator (tPA) were cloned from cell lines. The DNAs were connected via linkers and incorporated into the newly generated expression vector pCG-F1 (FIG. 5A).

The fusion protein anti-CD31-linker-K2P was expressed in E. coli from inclusion bodies and purified by affinity chromatography. The molecular weight of the expressed fusion protein was determined to be 70 kDa by visualization on SDS electrophoresis gels. The identity of the protein was confirmed by western blotting (FIG. 5).

### Example 3

### Fibrinolytic Activity of Anti-CD31-scFv-tPA

Fibrinolytic activity was assessed by a chromogenic assay, which measures the ability of the sample to cleave plasminogen to plasmin. The resulting plasmin is then measured via conversion of a chromogenic substrate specific for plasmin by spectrophotometry (FIG. 6).

### Example 4

### Cloning and Expression of Anti-PECAM scFv-scuPA

Reagents and cell lines: All chemicals were obtained from Sigma (St Louis, Mo.), unless otherwise specified. Drosophila S2 cells, pMT/Bip/V5 vector and the generation of a plasmid containing urokinase were described previously (Bdeir et al. Blood 2003 102:3600-3608). Drosophila serum-free medium was from Invitrogen (Carlsbad, Calif.). PCR core kit and Rapid DNA ligation kit were purchased from Roche (Basel, Switzerland). Endonucleases were obtained from New England Biolabs Inc. (Beverly, Mass.).

390 scFv was amplified from pww152 containing 390 scFv for cloning into the expression plasmid pMT/Bip/V5 using the upstream primer sen390 (5′-GGACTAGTCAGGTTACTCTGAAAGCGTCTGGCCC-3′; SEQ ID NO:1), which introduces a restriction site for SpeI at the 5′ end, and the downstream primer rev390 (5′-ATAAGAATGCGGCCGCGCCGGAAGAGCTACTACCCGATGAGGAAGAACGCAA TTCCACCTTGG-3′; SEQ ID NO:2), which appends the sequence of a short peptide linker (Ser₄Gly)₂ and a NotI restriction site.

Lmw-scuPA (Leu144-Leu411) was amplified with the primers senUK (5′-ATAAGAATGCGGCCGCATTAAAATTTCAGTGTGGCC-3′; SEQ ID NO:3), which introduces a NotI restriction site at the 5′ end, and downstream revUK (5′-CCGCTCGAGTCAGAGGGCCAGGCCATTC-3′; SEQ ID NO:4) to introduce an XhoI restriction site at the 3′ end. The 390 scFv-lmw scuPA construct was assembled as follows: first, two PCR products were purified and digested with SpeI, NotI and NotI, XhoI, respectively. Second, the two digested fragments were ligated and cloned into SpeI and XhoI sites of the drosophila expression vector pMT/Bip/V5. Successful cloning was confirmed by restriction analysis of recombinant plasmids and by automated nucleotide sequencing.

Drosophila S2 cells were co-transfected with the pMT-BD1 plasmid and pCoBlast (Invitrogen, Carlsbad, Calif.) at the ratio (w/w) of 19:1, and stable transfectants were established by adding blasticidin (25 μg/ml). Anti-PECAM scFv-scuPA, wild type scuPA, and active site mutant scuPA-Ser³⁵⁶Ala were expressed using the Drosophila Expression System (Invitrogen, Carlsbad, Calif.) and purified from cell media as previously described by Bdeir et al. (Blood 2003 102:3600-3608).

### Example 5

### Biochemical Characterization of Anti-PECAM scFv-uPA

The size and homogeneity of the fusion protein was analyzed on 10-15% gradient SDS/PAGE with or without addition of plasmin (molar ratio of 7.5%). Conversion to its two-chain derivative was determined after treatment with 50 mM DTT. For western blot analysis, fractionated proteins were electrotransferred to a PVDF membrane (Invitrogen, Carlsbad, Calif.) and unspecific binding was blocked with Tris (tris(hydroxymethyl)aminomethane)-buffered saline (pH=7.5) containing 10% non-fat milk powder and 0.1% Tween-20. A rabbit antibody against human uPA (American Diagnostica. Inc., Stamford, Conn.) served as the primary antibody. The secondary antibody was conjugated with peroxidase (Jackson Immunoresearch Laboratories, West Grove, Pa.) and the antigen-antibody complex was detected with ECL Plus (Amersham Biosciences, Piscataway, N.J.).

### Example 6

### Protein Modifications

For in vitro assays (Examples 9 and 10), the purified scFV-uPA fusion protein was biotinylated with Biotin-LC-NHS ester (Pierce, Rockford, Ill.) as previously described by Muzykantov et al. (Proc Natl Acad Sci USA. 1999; 96:2379-2384). For in vivo experiments (Examples 12 and 13), proteins were radiolabeled with ¹²⁵I-Na (Perkin Elmer, Wellesley, Mass.) using Iodogen (Pierce, Rockford, Ill.).

### Example 7

### Immunofluorescence Microscopy

REN cells, a human mesothelioma cell line previously isolated in accordance with procedures described by Smythe et al. (Cancer Res. 1994 54:2055-2059) were grown in RPMI1640 supplemented with 10% FBS and 2 mM L-glutamine (R10 media) containing 10,000 U penicillin and 10,000 U streptomycin. REN cells transfected with full-length mouse PECAM (REN-PECAM cells) have been previously described by Scherpereel et al. FASEB J. 2001 15:416-426.

REN cells transfected with cDNA encoding murine PECAM-1 were used to study the binding activity of 390 scFv-lmw scuPA. Untransfected REN cells served as the cell type control. Cells were seeded in 8-well chamber slides at a density of 1×10⁵/mL. After blocking with 5% bovine serum albumin/phosphate buffered saline (BSA/PBS) and 5 μg/mL scuPA to eliminate background signals, cells were incubated with 25 μg/mL biotinylated fusion protein at 4° C. for 2 hours and then with fluorescein-conjugated streptavidin (Calbiochem, Darmstadt, Germany) after fixation with ice-cold paraformaldehyde. Staining was visualized at 40× magnification.

### Example 8

### Cell-Bound ELISA

REN cells transfected with murine PECAM (see Example 7) were seeded in 48-well plates, fixed with ice-cold methanol and blocked with 5% BSA/PBS and 1 μg/mL scuPA. Various concentrations of biotinylated fusion proteins were added. After washing with PBS, cells were further incubated with peroxidase-conjugated streptavidin (Pierce, Rockford, Ill.). The colorimetric reaction was carried out with OPD substrate (Sigma, St Louis, Mo.), and absorbance at 490 nm was measured. A competition ELISA was used to determine the specificity of the fusion protein. Serial dilutions of P-390 monoclonal antibody were mixed with 20 μg/mL 390 scFv-lmw scuPA and incubated with methanol-fixed cells. Signals were developed as described above.

### Example 9

### Urokinase Activity

SPECTROZYME UK chromogenic substrate, plasmin and standard low molecular weight two-chain urokinase (lmw-tcuPA) were from American Diagnostica Inc. (Stamford, Conn.). Plasmin was added to a solution containing 0.2 μM purified fusion protein at different molar ratios (1, 2.5, 5, 7.5%). At various times thereafter, a chromogenic assay was performed by adding Spectrozyme® UK in assay buffer (50 mM Tris-HCl, 0.01% Tween 80 and 10 kIU/mL aprotinin, pH 8.5). The same range of concentrations of plasmin was incubated with substrate as a control. The amidolytic activity was determined by comparing the absorbance at 405 nm with that obtained with low molecular weight two-chain uPA standards. Fibrinolysis using fibrin-coated plates was performed as previously described by Muzykantov et al. (J Pharmacol Exp Therap. 1996 279:1026-1034). Briefly, 5 mg/mL human fibrinogen in PBS was mixed with thrombin (final concentration 1 μg/mL) and plasminogen (final concentration 250 nM). The mixture was poured onto the cover-lid of a 24-well plate to form a fibrin gel 5-mm in thickness. The indicated amounts of lmw-tcuPA, lmw-scuPA and the fusion protein were applied onto the surface of the fibrin clots and incubated at 37° C. for 5 hours. In other experiments, the amidolytic activity of each uPA preparation incubated with REN/mPECAM-1 cells was assayed by adding Spectrozyme® UK substrate as described above, but 1 μg/mL catalytically inactive scuPA-Ser³⁵⁶Ala was used to abolish unspecific binding via the lmw-scuPA portion of the molecule. The specificity of the binding was demonstrated by a competition assay in which various amounts of anti-PECAM IgG were added to each sample containing 20 μg/mL fusion protein.

### Example 10

### Biodistribution of Fusion Protein and lmw-scuPA In Vivo

Male and female C57BL/B6 mice of ages six to ten weeks were used throughout this study, except where noted. A breeding pair of PECAM-1 null mice originally created in accordance with procedures described by Duncan et al. (J. Immunol. 1999 162:3022-3030) was obtained from Yale University. These mice were backcrossed for over 10 generations onto the C57BL/B6 background. All protocols were performed in accordance with National Institutes of Health guidelines and with the approval the University of Pennsylvania Animal Use Committee.

Cocktails containing different amounts of unlabeled protein and a trace amount of radiolabeled protein (0.25 μg) were injected intravenously into anesthetized mice. At the indicated time points, blood was drawn and mice were sacrificed. Organs of interests were harvested, rinsed, weighed, and the ¹²⁵I activity in tissues and blood was measured in a gamma counter. The parameters of targeting including percent of injected dose per gram tissue (% ID/g), organ-to-blood ratio, and the immunospecificity index (ISI) were calculated after subtracting residual radioactivity in tubes and syringes as described (Danilov et al. Am J Physiol Lung Cell Mol Physiol 2001; 280:L1235-L1347).

### Example 11

### Prophylactic Fibrinolysis in a Model of Pulmonary Embolism

Ten minutes after injection of thrombolytic agents, a suspension of radiolabeled fibrin emboli was injected into anesthetized mice through the jugular vein as described by Murciano et al. (Nat. Biotechnol. 2003 21:891-896; Am J Physiol Lung Cell Mol. Physiol. 2002 282:L529-L539). One hour later, mice were sacrificed and residual isotope was measured in the lungs. As in previous studies (Murciano et al. Nat. Biotechnol. 2003 21:891-896; Murciano et al. Am J Physiol Lung Cell Mol. Physiol. 2002 282:L529-L539) spontaneous dissolution of emboli in control mice at this time was ˜50%, which was subtracted from fibrinolysis after administration of thrombolytics. Clot lysis under different conditions was compared by one-way ANOVA. All data are presented as the mean±SEM of at least three separate experiments.

### Example 12

### Construction and Expression of scFv/uPA-T

Mutagenesis was performed using the QuickChange® Site-Directed Mutagenesis kit from Stratagene (La Jolla, Calif.) according to the manufacturer's protocol. Two oligomers used were UKTsen: 5′-GTG GCC AAA AGA CTC TGA GGC CCC GCA TTA TTG GGG GAG AAT TCA CCA CCA TC-3′ (SEQ ID NO:9) and UKTrev, 5′-GAT GGT GGT GAA TTC TCC CCC AAT AAT GCG GGG CCT CAG AGT CTT TTG GCC AC-3′ (SEQ ID NO:10), which correspond to the DNA sequence encoding amino acids Cys¹⁴⁸ to Ile¹⁶⁷, except for the deletion of six nucleotides encoding amino acids Phe¹⁵⁷ and Lys¹⁵⁸.

### Example 13

### Biochemical Characterization and Binding of scFv/uPA-T Fusion Protein

The size and homogeneity of purified scFv/uPA-T was analyzed using 12% SDS-PAGE with or without addition of thrombin (150 nM). Conversion to its two-chain derivative was determined in the presence of 50 mM dithiothreitol (DTT). cDNA encoding the extracellular domain of mouse PECAM (amino acids Glu¹⁸-Lys⁵⁹⁰) was obtained by the reverse transcription polymerized chain reaction (RT-PCR) using mouse lung tissue total RNA. A FLAG affinity tag was fused to the N-terminus and subcloned into the BglII and NotI sites in the pMT/Bip vector. Stable cell lines were generated. Soluble mouse PECAM was purified from secreted supernatant using M2 anti-FLAG affinity chromatography. Specific binding of scFv/uPA-T to soluble PECAM was measured by ELISA. Each well within a 96-well plate was coated with 5 μg/ml soluble mouse PECAM protein overnight, the unbound sites were blocked with PBS containing 5% bovine serum albumin (BSA) at 37° C. for 1 h and various dilutions of scFv/uPA-T or WT lmw-scuPA were added for 1 hour. Unbound protein was removed by washing with PBS and 1 μg/ml anti-uPA monoclonal antibody in PBS containing 1% BSA was added. The wells were washed, horseradish peroxidase conjugated anti-mouse IgG was added, the wells washed again, TMB substrate was added and the optical density at 450 nm (OD450) was measured. A competition ELISA was used to demonstrate the specificity of binding. Purified scFv/uPA-T (10 μg/ml) mixed with various amounts of parental anti-PECAM IgG was incubated with the mouse PECAM-coated wells and the ELISA was performed as described above.

### Example 14

### Thrombin Mediated Release of PECAM-Bound scFv/uPA-T Fusion Protein

Purified scFv/uPA-T (25 μg/ml) was added to each well of a mouse PECAM-coated 96-well plate for 2 hours at 37° C. and washed with PBS. Thrombin (150 nM) was added for 2 hours at 37° C., the wells were washed and bound fusion protein was measured by ELISA as described above.

### Example 15

### Enzymatic Activity of scFv/uPA-T

scFv/uPA-T and lmw-scuPA were incubated with different amounts of thrombin or plasmin for 1 hour at 37° C. The resultant amidolytic activity was assayed in activity buffer (50 mM Tris, pH 7.5, 30 KIU/ml aprotinin and 30 U/ml hirudin). To detect protease activity, 1 ng of non-reduced or reduced fusion protein or thrombin-treated protein was separated on 10% SDS-PAGE containing 1% non-fat milk and 20 μg/ml plasminogen and renatured by adding 2.5% Triton X-100 in PBS for 30 min. The gel was transferred to zymogram developing buffer and stained with Simplyblue™ safestain. Fibrinolysis was measured using a plate assay. To measure the enzymatic activity of PECAM-associated scFv/uPA-T, various concentrations of scFv/uPA-T were added to mouse PECAM-coated wells for 1 hour at 37° C., 30 nM thrombin was added and OD405 was assayed as above.

### Example 16

### Enzyme Inhibitor Assay with scFv/uPA-T

Formation of SDS-stable complexes between scFv/uPA-T and PAI-1 was assessed by Western blot with an anti-uPA monoclonal antibody. Native or thrombin-activated scFv/uPA-T (10 ng) was incubated with different molar ratios of PAI-1 for 30 minutes at 37° C. and to the proteins was allowed to migrate on a 4-12% gradient SDS-PAGE. The proteins were transferred to a nitrocellulose membrane subsequently blocked with 5% non-fat milk and immunoblotting was performed with anti-uPA.

### Example 17

### In Vivo Biodistribution of scFv/uPA-T and scuPA

Male C57BL/B6 mice, 6-10 weeks of age were used throughout. All protocols were performed in accordance with National Institutes of Health guidelines and with the approval of the University of Pennsylvania Animal Use committee. Proteins were radiolabeled with ¹²⁵I-Na and tissue uptake was determined. In particular, cocktails containing 0.2 nmoles of unlabeled proteins and trace amounts of radiolabeled proteins were injected via the tail vein. At the indicated time points, blood was drawn and the mice were sacrificed. Organs of interests were harvested, washed, weighed, tissue radioactivity was measured, and the percentage of injected dose per gram tissue (% ID/g) was calculated.

### Example 18

### Stability of scuPA, scFv/uPA-T in Mouse Plasma

Various amounts of lmw-scuPA and scFv/uPA-T were incubated in 0.25 ml of citrated-pooled mouse plasma at 37° C. for 3 hours and the concentration of fibrinogen was measured as described by Gulledge et al. (Thromb Haemost 2001 86:511-516). Briefly, each well in 96-well plates was coated with 2 μg/ml rat anti-mouse fibrinogen antibody, unreactive sites blocked with 5% PBS-BSA and incubated with serially diluted mouse plasma for 1 hour at 37° C. Wells were incubated sequentially with 1 μg/ml biotinylated rat anti-mouse fibrinogen antibody, streptavidin-peroxidase and TMB substrate, and the OD450 was measured. Fibrinogen concentration was determined by comparison with a standard curve based on purified mouse fibrinogen. The plasma concentration fibrinogen in mice given an equal volume of saline was used as the baseline. To measure protein stability in vivo, 2 nmoles of lmw-scuPA, scFv/uPA-T or an equal volume (150 μl) of saline were injected intravenously. After 1 hour, 300 μl of whole blood was extracted from the jugular vein over 3.2% citrate solution in a 9:1 final ratio.

### Example 19

### Mouse Thrombosis Model

The model of thrombin-mediated thrombosis model was induced by thromboplastin as described by Leon et al. Circulation 2001 103:718-723). To quantify fibrinolysis, fibrin/fibrinogen deposition in lungs was measured as described by Lawson et al. (J Clin Invest 1997 99:1729-1738). Briefly, scFv/uPA-T (300 μg), equal molar amount of lmw-scuPA or scFv/uPA was injected intravenously. After 0.5 or 3 hours, Thromboplastin (85 μl/kg) pre-mixed with ¹²⁵I-fibrinogen (150,000 cpm was injected through the jugular vein. Ninety minutes later, mice were given heparin (400 U/mouse), sacrificed immediately, the lungs were harvested, washed, and residual isotope was measured. The extent of fibrinolysis was calculated by comparing residual lung radioactivity in PA-treated mice to control mice injected with saline.

### Example 20

### Data Analysis

All data are presented as the mean±s.e.m. of at least three separate experiments. Differences between groups were tested for statistical difference using Student's t-test or analysis of variance (ANOVA). P<0.05 was considered statistically different.

1. (canceled)

2. The method of claim 20 wherein said anti-thrombotic molecule has fibrinolytic, anticoagulant or platelet inhibiting activity.

3-4. (canceled)

5. The method of claim 20 wherein said anti-thrombotic molecule activates a precursor of an anti-coagulant to an active anti-coagulant.

6. The method of claim 20 wherein said anti-thrombotic molecule comprises a plasminogen activator or active fragment thereof or an anticoagulant.

7. The method of claim 6 wherein the plasminogen activator comprises tissue plasminogen activator, urokinase, tenectase, retavase, streptokinase or staphylokinase or an active fragment thereof.

8. The method of claim 20 wherein said anti-thrombotic molecule is thrombomodulin.

9. The method of claim 20 wherein the anticoagulant comprises protein C or activated protein C.

10. The method of claim 20 wherein said anti-thrombotic molecule is activated locally at a site of a pathological process by a pathological factor presented at the site of the pathological process.

11. The method of claim 10 wherein the pathological factor is a component of blood coagulation.

12. The method of claim 10 wherein the site of the pathological process is a site of active thrombosis and the pathological factor is thrombin.

13. The method of claim 10 wherein the pathological factor is a protease.

14. The method of claim 20 wherein said ligand or said anti-thrombotic molecule comprises a natural or artificial protease cleavage site for activation or release.

15. The method of claim 20 wherein said fusion protein is a recombinant fusion protein.

16. The method of claim 20 wherein said fusion protein is delivered in a pharmaceutically acceptable vehicle.

17. A method for inhibiting coagulation in a selected vascular bed of an animal comprising administering to said animal a fusion protein comprising a ligand which specifically binds to a luminal surface of vascular endothelium linked to an anti-thrombotic molecule, wherein the ligand comprises a single chain antigen-binding domain (scFv) of a monoclonal antibody that specifically binds to PECAM-1 or ICAM-1.

18. A method for dissolving blood clots in a selected vascular bed of an animal diagnosed as being predisposed or at high risk for thrombosis or thrombosis recurrence comprising prophylactically administering to said animal a fusion protein comprising a ligand which specifically binds to a luminal surface of vascular endothelium linked to an anti-thrombotic molecule, wherein the ligand comprises a single chain antigen-binding domain (scFv) of a monoclonal antibody that specifically binds to PECAM-1 or ICAM-1.

19. (canceled)

20. A method for enhancing effects of an anti-thrombotic molecule in an animal comprising administering to said animal a fusion protein comprising a ligand which specifically binds to a luminal surface of vascular endothelium linked to an anti-thrombotic molecule, wherein the ligand comprises a single chain antigen-binding domain (scFv) of a monoclonal antibody that specifically binds to PECAM-1 or ICAM-1.


SEQUENCE LISTING (11 sequences)

SEQ ID NO: 1 - DNA, 34 bp, Artificial
  synthetic
ggactagtcaggttactctgaaagcgtctggccc

SEQ ID NO: 2 - DNA, 63 bp, Artificial
  synthetic
ataagaatgcggccgcgccggaagagctactacccgatgaggaagaacgcaattccaccttgg

SEQ ID NO: 3 - DNA, 36 bp, Artificial
  synthetic
ataagaatgcggccgcattaaaatttcagtgtggcc

SEQ ID NO: 4 - DNA, 28 bp, Artificial
  synthetic
ccgctcgagtcagagggccaggccattc

SEQ ID NO: 5 - protein, 4 aa, Artificial
  synthetic
PRII

SEQ ID NO: 6 - protein, 13 aa, Artificial
  synthetic
SSSSGSSSSGAAA

SEQ ID NO: 7 - protein, 6 aa, Artificial
  synthetic
MYPRGN

SEQ ID NO: 8 - protein, 3 aa, Artificial
  synthetic
PRA

SEQ ID NO: 9 - DNA, 53 bp, Artificial
  synthetic
gtggccaaaagactctgaggccccgcattattgggggagaattcaccaccatc

SEQ ID NO: 10 - DNA, 53 bp, Artificial
  synthetic
gatggtggtgaattctcccccaataatgcggggcctcagagtcttttggccac

SEQ ID NO: 11 - protein, 15 aa, Artificial
  synthetic
GGGGSGGGGSGGGGS